(19) United States
(12) Patent Application Publication
(10) Pub. No.: US 2011/0011742 A1
(43) Pub. Date: Jan. 20, 2011

(54) METHOD FOR THE PURIFICATION OF BIOLOGICAL MACROMOLECULES

(76) Inventors: William D. Mathers, Lake Oswego, OR (US); Zheng Ye, Portland, OR (US)

Correspondence Address: Klarquist Sparkman LLP, 121 SW Salmon Street, Suite 1600, Portland, OR, 97204, US

(21) Appl. No.: 12/920,567

(22) PCT Filed: Mar. 2, 2009
(86) PCT No.: PCT/US09/35689

§ 371 Date: Sep. 1, 2010

Related U.S. Application Data
(60) Provisional application No. 61/033,331, filed on Mar. 3, 2008.

(51) Int. Cl. G01N 27/447; G01N 33/561

(52) U.S. Cl. 204/462

(57) ABSTRACT

Disclosed are methods and devices for electrophoretic separation of free biomolecules from molecular complexes comprising biomolecules of interest during which inhomogeneity in the molecular complexes is masked. The methods can include loading a sample which contains free biomolecules and the complexes that include biomolecules of interest to a proximal end of an electrophoresis gel. The sample is electrophoresed for a period of time sufficient for the free biomolecules to elute off a distal end of the gel while the molecular complexes that include the biomolecules of interest are retained in the electrophoresis gel, thereby separating the molecular complexes from the free biomolecules. The direction of electrophoresis is reversed and the sample is electrophoresed for a period of time sufficient for the molecular complexes of bound biomolecules of interest to elute from the proximal end of the electrophoresis gel.

### CROSS REFERENCE TO RELATED APPLICATION

This application claims the benefit of U.S. Provisional Application 61/033,331, filed Mar. 3, 2008, which is incorporated by reference herein in its entirety.

### FIELD

This disclosure concerns methods for the electrophoretic purification of biological macromolecules and devices for such methods.

### BACKGROUND

Electrophoresis is a method used to separate and analyze biological macromolecules, including proteins, DNA, RNA, and complexes of these molecules. Numerous electrophoretic techniques have been developed including capillary electrophoresis, gel electrophoresis, paper electrophoresis, and immunoelectrophoresis.

A common electrophoretic method is gel electrophoresis. In gel electrophoresis, a gel is formed using compounds such as agarose or polyacrylamide. A mixture containing the desired compound(s) is placed (or loaded) onto one end of the gel, and the gel is then placed contact with a liquid buffer. This liquid buffer contains salts, which when dissolved from ions within the buffer.

Biological molecules are typically charged, for example when contacted with electrophoresis buffer. For example, DNA is negatively charged in common electrophoresis buffers due to the phosphate groups in its backbone. Therefore, when electric current is applied to the ends of the gel, the biological molecules move through the gel from one end to the other. Depending on their size, shape, and charge, some molecules move through the gel faster than others. As the mixture moves through the gel, molecules of one size, shape, or charge are separated from those of a different size, shape, and/or charge. The speed at which the molecules pass through the gel and the separation between the various types of molecules also depends on the concentration and type of the gel. In general, molecules pass through thicker gels slower than they do through thinner gels, though thicker gels typically provide better separation.

The gels can be run either horizontally and/or vertically. When run horizontally, the gel is generally submerged in the buffer. The sample mixture is loaded at one end and run to the other end. When the gel is run vertically, the sample mixture is loaded at the top of the gel and run towards the bottom. A vertical gel is generally not submerged in buffer. Rather it is run on an electrophoresis apparatus that contains a buffer chamber at the top and the bottom of the gel.

### SUMMARY

Disclosed are methods for electrophoretic separation of free biomolecules from molecular complexes that include biomolecules of interest. In some examples the method results in the masking of inhomogeneity. Methods can include loading a sample, which contains free biomolecules and complexes that include biomolecules of interest, to the proximal end of an electrophoresis gel. The sample is then electrophoresed, for example by application of an electric current, for a period of time sufficient for the free biomolecules to elute from the distal end of the electrophoresis gel while the molecular complexes that include the biomolecules of interest are retained in the electrophoresis gel. This results in the separation of the molecular complexes from the free biomolecules. The direction of electrophoresis then is reversed and the sample is electrophoresed for a period of time sufficient for the molecular complexes of bound biomolecules of interest to elute from the proximal end of the electrophoresis gel. In some examples, reversing direction of electrophoresis is accomplished by reversing the orientation of the electrophoresis gel in an electrophoresis apparatus. In some examples, reversing direction of electrophoresis is accomplished by reversing the polarity of an electrophoresis apparatus, for example by reversing the current. Reversing the direction of electrophoresis masks inhomogeneity in the complexes that include biomolecules of interest. Thus, also provided are methods of masking inhomogeneity of one or both members of a specific binding pair that includes a biomolecule of interest during an electrophoretic purification of the specific binding pair.

In some examples, the methods also include collecting the molecular complexes that include biomolecules of interest, for example for use in subsequent analysis, such as microarray analysis. In some examples, the methods also include collecting the free biomolecules, for example for subsequent analysis.

The disclosed methods can be used with any material compatible with electrophoresis, for example polysaccharides (such as hydroxyethyl cellulose, hydroxypropylmethyl cellulose, methyl cellulose, agarose, hydroxyethyl agarose, galactomannan, dextran or a combinations thereof) crosslinked polymers (such as the free radical polymerization reaction product of a mixture comprising at least one monomer and at least one cross-linker sufficient to cross-link the monomer. In some examples, the monomer includes acrylamide, N-methylacrylamide, N,N-dimethylacrylamide, N-(hydroxymethyl)acrylamide, diacetonacrylamide, N-hydroxypropylacrylamide N-hydroxypropylacrylamide, N-acryloyl-tris(hydroxymethyl)aminomethane, N-acryloyl-1-amino-1-deoxy-D-galactitol, or a combination thereof, and the cross-linker includes N,N′-methylenebisacrylamide, N,N′-propylenebisacrylamide, diacrylamide dimethylether, 1,2-diacrylamide ethyleneglycol, ethylenureabisacrylamide, ethylene diacrylate, N,N′-diallyltartardiamide, N,N′-bisacrylylcystamine, N,N′-1,2-dihydroxyethylene-bisacrylamide, N,N-bisacrylyl cystamine, trisacryloyl-hexahydrotriazine, dihydroxyethylene-bis-acrylamide, piperazine-di-acrylamide, or a combination thereof) or even combinations of polysaccharides and cross-linked polymers.

Devices for carrying out the disclosed methods are also provided.

### BRIEF DESCRIPTION OF THE DRAWINGS

FIG. 1 is a schematic of an exemplary electrophoresis apparatus 100.

FIG. 2A is a schematic cross-section of an exemplary tube gel apparatus 200 for separating biological molecules.

FIG. 2B is a schematic cross-section of an exemplary tube gel apparatus 200 for separating biological molecules showing the separate components.

FIG. 3A is a schematic cross-section of an exemplary tube gel apparatus 200 for separating biological molecules showing the loading of a sample containing free biomolecules and molecular complexes to the proximal end of the electrophoresis gel.

FIG. 3B is a schematic cross-section of an exemplary tube gel apparatus 200 for separating biological molecules showing the migration of free biomolecules and molecular complexes in an applied electric current.

FIG. 4 is a schematic cross-section of an exemplary tube gel apparatus 200 for separating biological molecules showing the collection of free biomolecules eluted from the gel while the molecular complexes remain in the gel.

FIG. 5A is a schematic cross-section of an exemplary tube gel apparatus 200 for separating biological molecules showing migration of the molecular complexes after the applied electric field is reversed relative to the orientation of the tube gel apparatus.

FIG. 5B is a schematic cross-section of an exemplary tube gel apparatus 200 for separating biological molecules showing collection of the molecular complexes (note that the orientation of the tube gel is 180° opposed to the orientation shown in FIG. 5A).

FIG. 6 is a digital image showing a gel shift analysis in which recombinant Sp1 protein binds to its specific probe YZ9, where the probe is labeled with IR Dye 700.

FIG. 7 is a digital image showing a column gel in which recombinant ER-alpha protein is mixed with its specific probe labeled with IR Dye 700, and in which the sample is loaded on the column gel and run for 30 minutes.

### SEQUENCE LISTING

The nucleic acid sequences listed in the accompanying sequence listing are shown using standard letter abbreviations for nucleotide bases. Only one strand of each nucleic acid sequence is shown, but the complementary strand is understood as included by any reference to the displayed strand.

SEQ ID NOS: 1 and 2 are exemplary nucleic acid probes for NF-kb.

SEQ ID NOS: 3 and 4 are exemplary nucleic acid probes for Sp1.

SEQ ID NOS: 5 and 6 are exemplary nucleic acid probes for ER-alpha.

SEQ ID NOS: 7-32 are exemplary nucleic acid probes for the analysis of Sp1 binding to DNA.

### DETAILED DESCRIPTION

### I. Terms

Unless otherwise noted, technical terms are used according to conventional usage. Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Rédei, Encyclopedic Dictionary of Genetics, Genomics, and Proteomics, 2nd Edition, 2003 (ISBN: 0-471-26821-6).

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a probe” includes single or plural probes and is considered equivalent to the phrase “comprising at least one probe.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Although methods and materials similar or equivalent to those described herein can be used in the practice or testing of the present disclosure, suitable methods and materials are described below. The materials, methods, and examples are illustrative only and not intended to be limiting.

To facilitate review of the various embodiments of this disclosure, the following explanations of specific terms are provided:

Antibody: A polypeptide ligand that includes at least a light chain or heavy chain immunoglobulin variable region and specifically binds an epitope of an antigen. Antibodies can include monoclonal antibodies, polyclonal antibodies, or fragments of antibodies.

The term “specifically binds” refers to, with respect to an antigen, the preferential association of an antibody or other ligand, in whole or part, with a specific polypeptide, such as a specific double-stranded DNA binding protein, for example a transcription factor, such as an activated transcription factor. A specific binding agent binds substantially only to a defined target. It is recognized that a minor degree of non-specific interaction may occur between a molecule, such as a specific binding agent, and a non-target polypeptide. Nevertheless, specific binding can be distinguished as mediated through specific recognition of the antigen. Although selectively reactive antibodies bind antigen, they can do so with low affinity. Specific binding typically results in greater than 2-fold, such as greater than 5-fold, greater than 10-fold, or greater than 100-fold increase in amount of bound antibody or other ligand (per unit time) to a target polypeptide, such as compared to a non-target polypeptide. A variety of immunoassay formats are appropriate for selecting antibodies specifically immunoreactive with a particular protein. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity.

Antibodies can include a heavy and a light chain, each of which has a variable region, termed the variable heavy (VH) region and the variable light (VL) region. Together, the VH region and the VL region are responsible for binding the antigen recognized by the antibody. This includes intact immunoglobulins and the variants and portions of them well known in the art, such as Fab′ fragments, F(ab)′2 fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes recombinant forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, Immunology, 3rd Ed., W.H. Freeman & Co. New York, 1997.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. These fused cells and their progeny are termed “hybridomas.” Monoclonal antibodies include humanized monoclonal antibodies.

Aptamer: Small nucleic acid or peptide molecules that bind a specific target molecule, such as a target biomolecule or biomolecule of interest.

Binding or stable binding: An association between two substances or molecules, such as the hybridization of one nucleic acid molecule to another or itself, the association of an antibody with a peptide, or the association of a protein with another protein (for example the binding of a transcription factor to a cofactor) or nucleic acid molecule (for example the binding of a transcription factor to a partially double-stranded nucleic acid probe).

Binding can be detected by any procedure known to one skilled in the art, such as by physical or functional properties of the molecular complex. For example, binding can be detected functionally by determining whether binding has an observable effect upon a biosynthetic process such as expression of a gene, DNA replication, transcription, translation, and the like.

Physical methods of detecting the binding of complementary strands of nucleic acid molecules, include but are not limited to, methods such as DNase I or chemical footprinting, gel shift and affinity cleavage assays, Northern blotting, dot blotting and light absorption detection procedures. For example, the method can involve detecting a signal, such as a detectable label, present on one or both nucleic acid molecules (or antibody or protein as appropriate).

The binding between an oligomer and its target nucleic acid can be characterized by the temperature (Tₘ) at which 50% of the oligomer is melted from its target. A higher (Tₘ) means a stronger or more stable complex relative to a complex with a lower (Tₘ).

Biomolecule: A molecule that can be derived from a living organism, such as a polypeptide, a carbohydrate, a lipid, a small molecule and the like. In some examples, with reference to biomolecular polymers, such as polypeptides and nucleic acids, the sequence of polymer need not be found in nature, but can be produced from monomer building blocks, such as nucleotides and amino acids. In addition, the monomers need not be found in nature. A biomolecule can be obtained from organisms live or dead, for example nucleic acids polypeptides, carbohydrates, lipids, small molecules and the like isolated from an organism. Biomolecules also can be artificially produced molecules, such as recombinant polypeptides, nucleic acids, carbohydrates, lipids, small molecules and the like. For example, biomolecules can be produced synthetically or recombinantly. In some examples, a biomolecule is a nucleic acid, such as a RNA, DNA or a combination thereof. In some examples, a biomolecule is a peptide, such as a protein.

Binding site: A region on a protein, nucleic acid (such as DNA, RNA, or a combination thereof) to which other molecules stably bind. In one example, a binding site is the site on a DNA molecule, such as a partially double-stranded nucleic acid probe, that a double-stranded DNA binding protein, such as a transcription factor binds (referred to as a transcription factor binding site).

Complementarity and percentage complementarity: A double-stranded DNA or RNA strand includes of two complementary strands of base pairs (or one strand with a hairpin). Complementary binding occurs when the base of one nucleic acid molecule forms a hydrogen bond to the base of another nucleic acid molecule. Normally, the base adenine (A) is complementary to thymidine (T) and uracil (U), while cytosine (C) is complementary to guanine (G). For example, the sequence 5′-ATCG-3′ of one ssDNA molecule can bond to 3′-TAGC-5′ of another ssDNA to form a dsDNA. In this example, the sequence 5′-ATCG-3′ is the reverse complement of 3′-TAGC-5′.

Nucleic acid molecules can be complementary to each other even without complete hydrogen-bonding of all bases of each molecule. For example, hybridization with a complementary nucleic acid sequence can occur under conditions of differing stringency in which a complement will bind at some but not all nucleotide positions.

Molecules with complementary nucleic acids form a stable duplex or triplex when the strands bind, (hybridize), to each other by forming Watson-Crick, Hoogsteen or reverse Hoogsteen base pairs. Stable binding occurs when an oligonucleotide molecule remains detectably bound to a target nucleic acid sequence under the required conditions.

Complementarity is the degree to which bases in one nucleic acid strand base pair with the bases in a second nucleic acid strand. Complementarity is conveniently described by percentage, that is, the proportion of nucleotides that form base pairs between two strands or within a specific region or domain of two strands. For example, if 10 nucleotides of a 15-nucleotide oligonucleotide form base pairs with a targeted region of a DNA molecule, that oligonucleotide is said to have 66.67% complementarity to the region of DNA targeted.

In the present disclosure, “sufficient complementarity” means that a sufficient number of base pairs exist between an oligonucleotide molecule and a target nucleic acid sequence to achieve detectable binding. When expressed or measured by percentage of base pairs formed, the percentage complementarity that fulfills this goal can range from as little as about 50% complementarity to full (100%) complementary. In general, sufficient complementarity is at least about 50%, for example at least about 75% complementarity, at least about 90% complementarity, at least about 95% complementarity, at least about 98% complementarity, or even at least about 100% complementarity.

A thorough treatment of the qualitative and quantitative considerations involved in establishing binding conditions that allow one skilled in the art to design appropriate oligonucleotides for use under the desired conditions is provided by Beltz et al. Methods Enzymol. 100:266-285, 1983, and by Sambrook et al. (ed.), Molecular Cloning: A Laboratory Manual, 2nd ed., vol. 1-3, Cold Spring Harbor Laboratory Press, Cold Spring Harbor, N.Y., 1989.

Contacting: Placement in direct physical association, for example both in solid form and/or in liquid form (for example the placement of a probe in contact with a sample or an electrophoresis gel in contact with a solution, such as an electrophoresis buffer). In some examples, contacting can occur in vitro with isolated cells or substantially cell-free extracts, such as nuclear extracts.

Corresponding: The term “corresponding” is a relative term indicating similarity in position, purpose, or structure. For example, a nucleic acid sequence corresponding to a gene promoter indicates that the nucleic acid sequence is similar to the promoter found in an organism.

Cross-linked polymer: A three-dimensional network formed from the chemical reaction of monomers and a cross-linker.

Double-stranded nucleic acid binding protein: A protein that specifically binds to regions of double-stranded nucleic acids, such as duplex DNA, for example the double-stranded region of a partially double-stranded nucleic acid probe. Transcription factors are particular examples of double-stranded nucleic acid binding proteins, as are sigma factors in prokaryotic organisms.

Emission or emission signal: The light of a particular wavelength generated from a source. In particular examples, an emission signal is emitted from a fluorophore after the fluorophore absorbs light at its excitation wavelengths.

Electromagnetic radiation: A series of electromagnetic waves that are propagated by simultaneous periodic variations of electric and magnetic field intensity, and that includes radio waves, infrared, visible light, ultraviolet light, X-rays and gamma rays. In particular examples, electromagnetic radiation is emitted by a laser, which can possess properties of monochromaticity, directionality, coherence, polarization, and intensity. Lasers are capable of emitting light at a particular wavelength (or across a relatively narrow range of wavelengths), for example such that energy from the laser can excite one fluorophore not another fluorophore.

Electrophoresis: The process of separating a mixture of charged molecules based on the different mobility of these charged molecules in response to an applied electric current. A particular type of electrophoresis is gel electrophoresis. The mobility of a molecule is generally related to the characteristics of the charged molecule, such as size, shape, and surface charge amongst others. The mobility of a molecule also is influenced by the electrophoretic medium, for example the composition of the electrophoresis gel. For example, when the electrophoretic medium is cross-linked acrylamide (polyacrylamide) increasing the percentage if acrylamide in the gel reduces the size of the resulting pores in the gel and retards the mobility of a molecule relative to a gel with a lower percentage of acrylamide (larger pore size). Gel electrophoresis can be performed for analytical purposes, but can be used as a preparative technique to partially purify molecules prior to use of other methods, such as mass spectrometry, PCR, cloning, DNA sequencing, array analysis, and immuno-blotting.

Excitation or excitation signal: The light of a particular wavelength necessary and/or sufficient to excite an electron transition to a higher energy level. In particular examples, an excitation is the light of a particular wavelength necessary and/or sufficient to excite a fluorophore to a state such that the fluorophore will emit a different (such as a longer) wavelength of light then the wavelength of light from the excitation signal.

For a period of time sufficient: A phrase used to describe a period of time in which a desired activity occurs, for example the time it takes for a biomolecule or a complex of biomolecules (molecular complex or biomolecular complex) to pass through a portion of an electrophoresis gel under the influence of an applied electric current. For example, the time it takes for a free biomolecule to travel from a proximal end of a gel to the distal end and elute from the distal end of an electrophoresis gel. In another example, it is the time it takes for a molecular complex, such as a complex containing a biomolecule of interest, to pass from a position within an electrophoresis gel to the end of the electrophoresis gel, and elute from that end. It is appreciated by those of ordinary skill in the art that the period of time sufficient for a free biomolecule to pass from one end of an electrophoresis gel to the other, or the time it takes for a molecular complex, such as a complex containing a biomolecule of interest, to pass from a position within an electrophoresis gel to the end of the electrophoresis gel, and elute the end, will depend on such factors as the composition of the gel, strength of the electric current, and the physical characteristics of the biomolecules or complexes. It is also appreciated that these time periods can be varied by the practitioner according to his or her needs.

Hairpin or nucleic acid hairpin: A nucleic acid structure formed from a single strand of nucleic acid. The strand exhibits self-complementarity, such that the nucleic acid hybridizes with itself, forming a loop at one end.

Hybridization: The ability of complementary single-stranded DNA or RNA to form a duplex molecule (also referred to as a hybridization complex). Nucleic acid hybridization techniques can be used to form hybridization complexes between a probe, such as the single-stranded portion of a partially double-stranded nucleic acid probe and an indexing probe.

Hybridization conditions resulting in particular degrees of stringency will vary depending upon the nature of the hybridization method and the composition and length of the hybridizing nucleic acid sequences. Generally, the temperature of hybridization and the ionic strength (such as the Na+ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11). The following is an exemplary set of hybridization conditions and is not limiting:

Very High Stringency (Detects Sequences that Share at Least 90% Identity)
Hybridization: 5×SSC at 65° C. for 16 hours
Wash twice: 2×SSC at room temperature (RT) for 15 minutes each
Wash twice: 0.5×SSC at 65° C. for 20 minutes each
High Stringency (Detects Sequences that Share at Least 80% Identity)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each
Low Stringency (Detects Sequences that Share at Least 50% Identity)
Hybridization: 6×SSC at RT to 55° C. for 16-20 hours
Wash at least twice: 2×-3×SSC at RT to 55° C. for 20-30 minutes each.

Isolated: An “isolated” biological component (such as a protein, a molecular complex, a nucleic acid probe, or free biomolecule) has been substantially separated or purified away from other biological components in the cell of the organism in which the component naturally occurs, for example, extra-chromatin DNA and RNA, proteins and organelles. The term also embraces nucleic acids and proteins prepared by recombinant expression in a host cell as well as chemically synthesized nucleic acids. It is understood that the term “isolated” does not imply that the biological component is free of trace contamination, and can include nucleic acid molecules, proteins or molecular complexes that are at least 50% isolated, such as at least 75%, 80%, 90%, 95%, 98%, 99%, or even 100% isolated.

Label: An agent capable of detection, for example by spectrophotometry, flow cytometry, or microscopy. For example, a label can be attached to a specific binding agent, such as an antibody or a protein, nucleic acid, and the like, thereby permitting detection of the specific binding agent or a biomolecule bound to the specific binding agent. Specific, non-limiting examples of labels include fluorophores, enzymatic linkages, and radioactive isotopes and nanoparticles, such as semiconductor nanocrystals. Methods for labeling are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

Masking inhomogeneity: Canceling out the differences in one or more physical properties of two or more molecules. In one non-limiting example, the physical property is molecular weight.

Molecular complex: Two or more molecules, such as biomolecules, that are stably bound together, for example a nucleic acid binding protein and a nucleic acid molecule, an antibody and an antigen, a ligand and receptor for the ligand, and the like. In some examples, a molecular complex is a transcription factor bound to a nucleic acid that has a binding site for the transcription factor.

Mutation: A change of the DNA sequence relative to the native or wild-type sequence, for example in a promoter of a gene. In some instances, a mutation will alter a characteristic of the DNA sequence, for example the binding of a double-stranded binding protein to the DNA sequence. Mutations include base substitution point mutations, deletions, and insertions as well as combinations thereof. Mutations can be introduced, for example by molecular biological techniques. In some examples, a mutation, such as a mutation in the promoter sequence of a gene, is introduced during synthesis of an oligonucleotide, such as an oligonucleotide that is part of a partially double-stranded nucleic acid probe.

Nucleic acid molecule (or sequence): A deoxyribonucleotide or ribonucleotide polymer including without limitation, cDNA, mRNA, genomic DNA, and synthetic (such as chemically synthesized) DNA or RNA or a combination of DNA and RNA. The nucleic acid molecule can be double stranded (ds) or single stranded (ss) or even partially double-stranded. Where single stranded, the nucleic acid molecule can be the sense strand or the antisense strand. Nucleic acid molecules can include natural nucleotides (such as A, T/U, C, and G), and can also include analogs of natural nucleotides. In one embodiment, a nucleic acid molecule is an aptamer.

Nucleotide: The fundamental unit of nucleic acid molecules. A nucleotide includes a nitrogen-containing base attached to a pentose monosaccharide with one, two, or three phosphate groups attached by ester linkages to the saccharide moiety.

The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U).

Nucleotides include those nucleotides containing modified bases, modified sugar moieties and modified phosphate backbones, for example as described in U.S. Pat. No. 5,866,336 to Nazarenko et al.

Examples of modified base moieties which can be used to modify nucleotides at any position on its structure include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N˜6-sopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl)uracil, and 2,6-diaminopurine.

Examples of modified sugar moieties which may be used to modify nucleotides at any position on its structure include, but are not limited to: arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

Oligonucleotide or “oligo”: Multiple nucleotides (that is, molecules including a sugar (for example, ribose or deoxyribose) linked to a phosphate group and to an exchangeable organic base, which is either a substituted pyrimidine (Py) (for example, cytosine (C), thymine (T) or uracil (U)) or a substituted purine (Pu) (for example, adenine (A) or guanine (G)). The term “oligonucleotide” as used herein refers to both oligoribonucleotides and oligodeoxyribonucleotides. Oligonucleotides can be obtained from existing nucleic acid sources (for example, genomic or cDNA), but are preferably synthetic (that is, produced by oligonucleotide synthesis).

Partially double-stranded nucleic acid probe: A nucleic acid probe that includes both a region that is single-stranded and a region or portion that is double-stranded. A partially double-stranded nucleic acid probe has a portion that is double-stranded and a portion that is single-stranded, wherein the double-stranded and single-stranded portions are connected, for example covalently linked. In some examples, the double-stranded portion includes a binding site for a double-stranded nucleic acid binding protein, such as a transcription factor.

Peptide/Protein/Polypeptide: All of these terms refer to a polymer of amino acids and/or amino acid analogs that are joined by peptide bonds or peptide bond mimetics. The twenty naturally occurring amino acids and their single-letter and three-letter designations are known in the art.

Polymerization: The reaction of monomer molecules together in a chemical reaction to form linear chains or a three-dimensional network of polymer chains. In one embodiment, the polymer polyacrylamide is formed from the polymerization of acrylamide in the presence of a cross-linker, which in some embodiments is N′,N-methylenebisacrylamide (BIS). Upon the introduction of catalyst, the polymerization of acrylamide and BIS proceeds via a free-radical mechanism. The most common system of catalytic initiation involves the production of free oxygen radicals by ammonium persulfate (APS) in the presence of the tertiary aliphatic amine N,N,N′,N′-tetramethylethylenediamine (TEMED) although other free radical generators can be employed.

Polysaccharide: A polymer of covalently linked sugars. The sugars making up the polysaccharide can by the same sugar or different sugars.

Promoter: An array of nucleic acid control sequences, which directs transcription of a nucleic acid. Typically, a eukaryotic a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription, such as specific DNA sequences that are recognized by proteins known as transcription factors.

In prokaryotes, a promoter is recognized by RNA polymerase and an associated sigma factor, which in turn are brought to the promoter DNA by an activator protein binding to its own DNA sequence nearby.

Reverse: To change direction opposite the initial direction. For example, if a biomolecule is traveling away from position A toward position B, reversing direction would mean that that the biomolecule is traveling away from position B toward position A. Reversing the direction of travel of a biomolecule in an electrophoresis gel can be accomplished by reversing the polarity of an electrophoresis apparatus, for example by reversing the direction of the current, while the gel remains static with respect to the electrophoresis apparatus, or reversing the orientation of the gel with respect to the applied electric current.

Sample: Any quantity of a substance that includes biomolecules (such as nucleic acid and proteins, for example double-stranded nucleic acid binding proteins) that can be used in a method disclosed herein. The sample can be a biological sample or can be extracted from a biological sample derived from humans, animals, plants, fungi, yeast, bacteria, tissue cultures, viral cultures, or combinations thereof.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Homologs or orthologs of nucleic acid or amino acid sequences possess a relatively high degree of sequence identity/similarity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. One indication that two nucleic acid molecules are closely related is that the two molecules hybridize to each other under stringent conditions. Stringent conditions are sequence-dependent and are different under different environmental parameters.

Target biomolecule or biomolecule of interest: A biomolecule about which information is desired. A target biomolecule or biomolecule of interest can be any molecule that is or once was part of a living organism or synthetically produced. In several non-limiting examples, a target biomolecule is a polypeptide, a nucleic acid, a ligand, or a small molecule. In one example, the information desired is location of the biomolecule on or within a cell, such as a cell in a biological sample. In another example, the information desired is the presence or absence of the biomolecule, for example in a sample, such as a biological sample. In another example, the information desired is the presence, absence, and/or location of the biomolecule in a gel, such as an electrophoreses gel.

Transcription factor: A protein that regulates transcription. In particular, transcription factors regulate the binding of RNA polymerase and the initiation of transcription. A transcription factor binds upstream or downstream to either enhance or repress transcription of a gene by assisting or blocking RNA polymerase binding. The term transcription factor includes both inactive and activated transcription factors.

Transcription factors are typically modular proteins that affect regulation of gene expression. Exemplary transcription factors include AAF, abl, ADA2, ADA-NF1, AF-1, AFP1, AhR, AIIN3. ALL-1, alpha-CBF, alpha-CP1, alpha-CP2a, alpha-CP2b, alphaHo, alphaH2-alphaH3, Alx-4, aMEF-2, AML1, AML1a, AML1b, AML1c, AML1DeltaN, AML2, AML3, AML3a, AML3b, AMY-1L, A-Myb, ANF, AP-1, AP-2alphaA, AP-2alphaB, AP-2beta, AP-2gamma, AP-3 (1), AP-3 (2), AP-4, AP-5, APC, AR, AREB6, Arnt, Arnt (774 M form), ARP-1, ATBF1-A, ATBF1-B, ATF, ATF-1, ATF-2, ATF-3, ATF-3deltaZIP, ATF-a, ATF-adelta, ATPF1, Barhl1, Barhl2, Barx1, Barx2, Bcl-3, BCL-6, BD73, beta-catenin, Bin1, B-Myb, BP1, BP2, brahma, BRCA1, Brn-3a, Brn-3b, Brn-4, BTEB, BTEB2, B-TFIID, C/EBPalpha, C/EBPbeta, C/EBPdelta, CACCbinding factor, Cart-1, CBF (4), CBF (5), CBP, CCAAT-binding factor, CCMT-binding factor, CCF, CCG1, CCK-1a, CCK-1b, CD28RC, cdk2, cdk9, Cdx-1, CDX2, Cdx-4, CFF, Chx10, CLIM1, CLIM2, CNBP, CoS, COUP, CP1, CP1A, CP1C, CP2, CPBP, CPE binding protein, CREB, CREB-2, CRE-BP1, CRE-BPa, CREMalpha, CRF, Crx, CSBP-1, CTCF, CTF, CTF-1, CTF-2, CTF-3, CTF-5, CTF-7, CUP, CUTL1, Cx, cyclin A, cyclin T1, cyclin T2, cyclin T2a, cyclin T2b, DAP, DAX1, DB1, DBF4, DBP, DbpA, DbpAv, DbpB, DDB, DDB-1, DDB-2, DEF, deltaCREB, deltaMax, DF-1, DF-2, DF-3, Dlx-1, Dlx-2, Dlx-3, DIx4 (long isoform), Dlx-4 (short isoform, Dlx-5, Dlx-6, DP-1, DP-2, DSIF, DSIF-p14, DSIF-p160, DTF, DUX1, DUX2, DUX3, DUX4, E, E12, E2F, E2F+E4, E2F+p107, E2F-1, E2F-2, E2F-3, E2F-4, E2F-5, E2F-6, E47, E4BP4, E4F, E4F1, E4TF2, EAR2, EBP-80, EC2, EF1, EF-C, EGR1, EGR2, EGR3, EIIaE-A, EIIaE-B, EIIaE-Calpha, EIIaE-Cbeta, EivF, EIf-1, Elk-1, Emx-1, Emx-2, Emx-2, En-1, En-2, ENH-bind. prot., ENKTF-1, EPAS1, epsilonF1, ER, Erg-1, Erg-2, ERR1, ERR2, ETF, Ets-1, Ets-1 deltaVil, Ets-2, Evx-1, F2F, factor 2, Factor name, FBP, f-EBP, FKBP59, FKHL18, FKHRL1P2, Fli-1, Fos, FOXB1, FOXC1, FOXC2, FOXD1, FOXD2, FOXD3, FOXD4, FOXE1, FOXE3, FOXF1, FOXF2, FOXG1a, FOXG1b, FOXG1c, FOXH1, FOXI1, FOXJ1a, FOXJ1b, FOXJ2 (long isoform), FOXJ2 (short isoform), FOXJ3, FOXK1a, FOXK1b, FOXK1c, FOXL1, FOXM1a, FOXM1b, FOXM1c, FOXN1, FOXN2, FOXN3, FOX01a, FOX01b, FOXO2, FOXO3a, FOXO3b, FOXO4, FOXP1, FOXP3, Fra-1, Fra-2, FTF, FTS, G factor, G6 factor, GABP, GABP-alpha, GABP-beta1, GABP-beta2, GADD 153, GAF, gammaCMT, gammaCAC1, gammaCAC2, GATA-1, GATA-2, GATA-3, GATA-4, GATA-5, GATA-6, Gbx-1, Gbx-2, GCF, GCMa, GCN5, GF1, GLI, GLI3, GR alpha, GR beta, GRF-1, Gsc, Gsc1, GT-IC, GT-IIA, GT-IIBalpha, GT-IIBbeta, H1TF1, H1TF2, H2RIIBP, H4TF-1, H4TF-2, HAND1, HAND2, HB9, HDAC1, HDAC2, HDAC3, hDaxx, heat-induced factor, HEB, HEB1-p67, HEB1-p94, HEF-1 B, HEF-1T, HEF-4C, HEN1, HEN2, Hesxl, Hex, HIF-1, HIF-1alpha, HIF-1beta, HiNF-A, HiNF-B, HINF—C, HINF-D, HiNF-D3, HiNF-E, HiNF—P, HIP1, HIV-EP2, Hlf, HLTF, HLTF (Met123), HLX, HMBP, HMG I, HMG I(Y), HMG Y, HMGI-C, HNF-1A, HNF-1B, HNF-1C, HNF-3, HNF-3alpha, HNF-3beta, HNF-3gamma, HNF4, HNF-4-alpha, HNF4alpha1, HNF-4-alpha2, HNF-4-alpha3, HNF-4-alpha4, HNF4gamma, HNF-6alpha, hnRNP K, HOX11, HOXA1, HOXA10, HOXA10 PL2, HOXA11, HOXA13, HOXA2, HOXA3, HOXA4, HOXA5, HOXA6, HOXA7, HOXA9A, HOXA9B, HOXB-1, HOXB13, HOXB2, HOXB3, HOXB4, HOXB5, HOXB6, HOXA5, HOXB7, HOXB8, HOXB9, HOXC10, HOXC11, HOXC12, HOXC13, HOXC4, HOXC5, HOXC6, HOXC8, HOXC9, HOXD10, HOXD11, HOXD12, HOXD13, HOXD3, HOXD4, HOXD8, HOXD9, Hp55, Hp65, HPX42B, HrpF, HSF, HSF1 (long), HSF1 (short), HSF2, hsp56, Hsp90, IBP-1, ICER-II, ICER-ligamma, ICSBP, Id1, Id1 H′, Id2, Id3, Id3/Heir-1, IF1, IgPE-1, IgPE-2, IgPE-3, IkappaB, IkappaB-alpha, IkappaB-beta, IkappaBR, II-1 RF, IL-6 RE-BP, 11-6 RF, INSAF, IPF1, IRF-1, IRF-2, ir1B, IRX2a, Irx-3, Irx-4, ISGF-1, ISGF-3, ISGF3alpha, ISGF-3gamma, 1s1-1, ITF, ITF-1, ITF-2, JRF, Jun, JunB, JunD, kappay factor, KBP-1, KER1, KER-1, Kox1, KRF-1, Ku autoantigen, KUP, LBP-1, LBP-1a, LBX1, LCR-F1, LEF-1, LEF-1B, LF-A1, LHX1, LHX2, LHX3a, LHX3b, LHX5, LHX6.1a, LHX6.1b, LIT-1, Lmo1, Lmo2, LMX1A, LMX1B, L-My1 (long form), L-My1 (short form), L-My2, LSF, LXRalpha, LyF-1, LyI-1, M factor, Mad1, MASH-1, Max1, Max2, MAZ, MAZ1, MB67, MBF1, MBF2, MBF3, MBP-1 (1), MBP-1 (2), MBP-2, MDBP, MEF-2, MEF-2B, MEF-2C (433 AA form), MEF-2C (465 AA form), MEF-2C (473 M form), MEF-2C/delta32 (441 AA form), MEF-2D00, MEF-2DOB, MEF-2DAO, MEF-2DA′0, MEF-2DAB, MEF-2DA′B, Meis-1, Meis-2a, Meis-2b, Meis-2c, Meis-2d, Meis-2e, Meis3, Meox1, Meox1a, Meox2, MHox (K-2), Mi, MIF-1, Miz-1, MM-1, MOP3, MR, Msx-1, Msx-2, MTB-Zf, MTF-1, mtTF1, Mxi1, Myb, Myc, Myc 1, Myf-3, Myf-4, Myf-5, Myf-6, MyoD, MZF-1, NC1, NC2, NCX, NELF, NER1, Net, NF III-a, NF III-c, NF III-e, NF-1, NF-1A, NF-1B, NF-1X, NF-4FA, NF-4FB, NF-4FC, NF-A, NF-AB, NFAT-1, NF-AT3, NF-Atc, NF-Atp, NF-Atx, NfbetaA, NF-CLEOa, NF-CLEOb, NFdeltaE3A, NFdeltaE3B, NFdeltaE3C, NFdeltaE4A, NFdeltaE4B, NFdeltaE4C, Nfe, NF-E, NF-E2, NF-E2 p45, NF-E3, NFE-6, NF-Gma, NF-GMb, NF-IL-2A, NF-IL-2B, NF-jun, NF-kappaB, NF-kappaB(-like), NF-kappaB1, NF-kappaB1, precursor, NF-kappaB2, NF-kappaB2 (p49), NF-kappaB2 precursor, NF-kappaE1, NF-kappaF2, NF-kappaF3, NF-MHCIIA, NF-MHCIIB, NF-muE1, NF-muE2, NF-muE3, NF-S, NF-X, NF-X1, NF-X2, NF-X3, NF-Xc, NF-YA, NF-Zc, NF-Zz, NHP-1, NHP-2, NHP3, NHP4, NKX2-5, NKX2B, NKX2C, NKX2G, NKX3A, NKX3A v1, NKX3A v2, NKX3A v3, NKX3A v4, NKX3B, NKX6A, Nmi, N-Myc, N-Oct-2alpha, N-Oct-2beta, N-Oct-3, N-Oct-4, N-Oct-5a, N-Oct-5b, NP-TCII, NR2E3, NR4A2, Nrf1, Nrf-1, Nrf2, NRF-2beta1, NRF-2gamma1, NRL, NRSF form 1, NRSF form 2, NTF, O2, OCA-B, Oct-1, Oct-2, Oct-2.1, Oct-2B, Oct-2C, Oct-4A, Oct4B, Oct-5, Oct-6, Octa-factor, octamer-binding factor, oct-B2, oct-B3, Otx1, Otx2, OZF, p107, p130, p28 modulator, p300, p38erg, p45, p49erg, -p53, p55, p55erg, p65delta, p67, Pax-1, Pax-2, Pax-3, Pax-3A, Pax-3B, Pax-4, Pax-5, Pax-6, Pax-6/Pd-5a, Pax-7, Pax-8, Pax-8a, Pax-8b, Pax-8c, Pax-8d, Pax-8e, Pax-8f, Pax-9, Pbx-1a, Pbx-1b, Pbx-2, Pbx-3a, Pbx-3b, PC2, PC4, PC5, PEA3, PEBP2alpha, PEBP2beta, Pit-1, PITX1, PITX2, PITX3, PKNOX1, PLZF, PO-B, Pontin52, PPARalpha, PPARbeta, PPARgamma1, PPARgamma2, PPUR, PR, PR A, pRb, PRD1-BF1, PRDI-BFc, Prop-1, PSEL P-TEFb, PTF, PTFalpha, PTFbeta, PTFdelta, PTFgamma, Pu box binding factor, Pu box binding factor (BJA-B), PU.1, PuF, Pur factor, R1, R2, RAR-alpha1, RAR-beta, RAR-beta2, RAR-gamma, RAR-gamma1, RBP60, RBP-Jkappa, Rel, RelA, RelB, RFX, RFX1, RFX2, RFX3, RFX5, RF-Y, RORalphal, RORalpha2, RORalpha3, RORbeta, RORgamma, Rox, RPF1, RPGalpha, RREB-1, RSRFC4, RSRFC9, RVF, RXR-alpha, RXR-beta, SAP-1a, SAP1b, SF-1, SHOX2a, SHOX2b, SHOXa, SHOXb, SHP, SIII-p110, SIII-p15, SIII-p18, SIM1, Six-1, Six-2, Six-3, Six-4, Six-5, Six-6, SMAD-1, SMAD-2, SMAD-3, SMAD-4, SMAD-5, SOX-11, SOX-12, Sox-4, Sox-5, SOX-9, Sp1, Sp2, Sp3, Sp4, Sph factor, Spi-B, SPIN, SRCAP, SREBP-1a, SREBP-1b, SREBP-1c, SREBP-2, SRE-ZBP, SRF, SRY, SRP1, Staf-50, STAT1alpha, STAT1beta, STAT2, STAT3, STAT4, STAT6, T3R, T3R-alpha1, T3R-alpha2, T3R-beta, TAF(I)110, TAF(I)48, TAF(I)63, TAF(II)100, TAF(II)125, TAF(II)135, TAF(II)170, TAF(II)18, TAF(II)20, TAF(II)250, TAF(II)250Delta, TAF(II)28, TAF(II)30, TAF(II)31, TAF(II)55, TAF(II)70-alpha, TAF(II)70-beta, TAF(II)70-gamma, TAF-I, TAF-II, TAF-L, Tal-1, Tal-1beta, Tal-2, TAR factor, TBP, TBX1A, TBX1 B, TBX2, TBX4, TBX5 (long isoform), TBX5 (short isoform), TCF, TCF-1, TCF-1A, TCF-1B, TCF-1C, TCF-1D, TCF-1E, TCF-1F, TCF-1G, TCF-2alpha, TCF-3, TCF-4, TCF-4(K), TCF-4B, TCF-4E, TCFbeta1, TEF-1, TEF-2, tel, TFE3, TFEB, TFIIA, TFIIA-alpha/beta precursor, TFIIA-alpha/beta precursor, TFIIA-gamma, TFIIB, TFIID, TFIIE, TFIIE-alpha, TFIIE-beta, TFIIF, TFIIF-alpha, TFIIF-beta, TFIIH, TFIIH*, TFIIH-CAK, TFIIH-cyclin H, TFIIH-ERCC2/CAK, TFIIH-MAT1, TFIIH-M015, TFIIH-p34, TFIIH-p44, TFIIH-p62, TFIIH-p80, TFIIH-p90, TFII-I, Tf-LF1, Tf-LF2, TGIF, TGIF2, TGT3, THRA1, TIF2, TLE1, TLX3, TMF, TR2, TR2-11, TR2-9, TR3, TR4, TRAP, TREB-1, TREB-2, TREB-3, TREF1, TREF2, TRF (2), TTF-1, TXRE BP, TxREF, UBF, UBP-1, UEF-1, UEF-2, UEF-3, UEF-4, USF1, USF2, USF2b, Vav, Vax-2, VDR, vHNF-1A, vHNF-1B, vHNF-1C, VITF, WSTF, WT1, WT11, WT1 I-KTS, WT1 I-de12, WT1-KTS, WT1-de12, X2BP, XBP-1, XW-V, XX, YAF2, YB-1, YEBP, YY1, ZEB, ZF1, ZF2, ZFX, ZHX1, ZIC2, ZID, ZNF174, amongst others.

An activated transcription factor is a transcription factor that has been activated by a stimulus resulting in a measurable change in the state of the transcription factor, for example a post-translational modification, such as phosphorylation, methylation, and the like. Activation of a transcription factor can result in a change in the affinity for a particular DNA sequence or of a particular protein, such as another transcription factor and/or cofactor.

Under conditions that permit binding: A phrase used to describe any environment that permits the desired activity, for example conditions under which two or more molecules, such as nucleic acid molecules and/or protein molecules, can bind. Such conditions can include specific concentrations of salts and/or other chemicals that facilitate the binding of molecules. In some examples, conditions that permit binding are similar to the conditions found in the nucleus of a cell, for example a eukaryotic cell or the cytoplasm of a prokaryotic cell. Such conditions can be simulated, for example by using a nuclear extract.

### II. Description of Several Embodiments

Electrophoresis can be used to the separate and analyze biomolecules, including proteins, DNA, RNA, and complexes of these biomolecules. Separation of these molecules within an electrophoresis gel is easily achieved; however, subsequent collection of the isolated molecules for further analysis can be inefficient and difficult. This is especially true when the individual biomolecules or complexes containing the biomolecules have different physical characteristics, such as molecular weights and/or charges. For example, in traditional gel electrophoresis, as a mixture biomolecules moves through the gel, molecules of one size, shape, or charge are separated from those of a different size, shape, and/or charge.

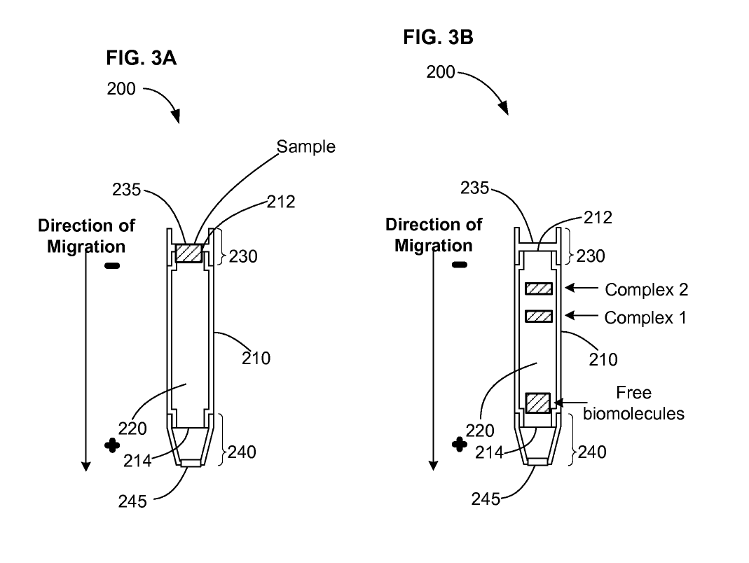

In gel electrophoresis one takes advantage of the fact that macromolecules having a different charge and size migrate with a different velocity (distance traveled per unit time) in a gel when influenced by an external electric current, and bands are thereby generated each containing a species of the different macromolecules, for example the macromolecules having the same charge or the same size. This is depicted in FIG. 3B in which the free biomolecules, complex 1, and complex 2 are separated across the length of the gel. A problem arises if one tries to collect the biomolecules of interest that have different molecular weights, for example complexes of biomolecules of interest that have different molecular weights, as they elute from the end of the electrophoresis gel. In such a situation, molecules of different molecular weights will to spread across a large volume of buffer. This leads significant waste of time and possible loss of the biomolecules of interest because of the need to concentrate the biomolecules of interest before additional use or analysis.

To overcome the problems with conventional gel electrophoretic separation and purification, methods for separating biomolecules of interest in a complex from free biomolecules is disclosed herein. While the methods are described with reference to the separation of complexes of biomolecules of interest, it will be appreciated by those of ordinary skill in the art that any group of large molecules could potentially be separated from a set of smaller molecules by the disclosed methods and devices.

### A. Exemplary Electrophoresis Device

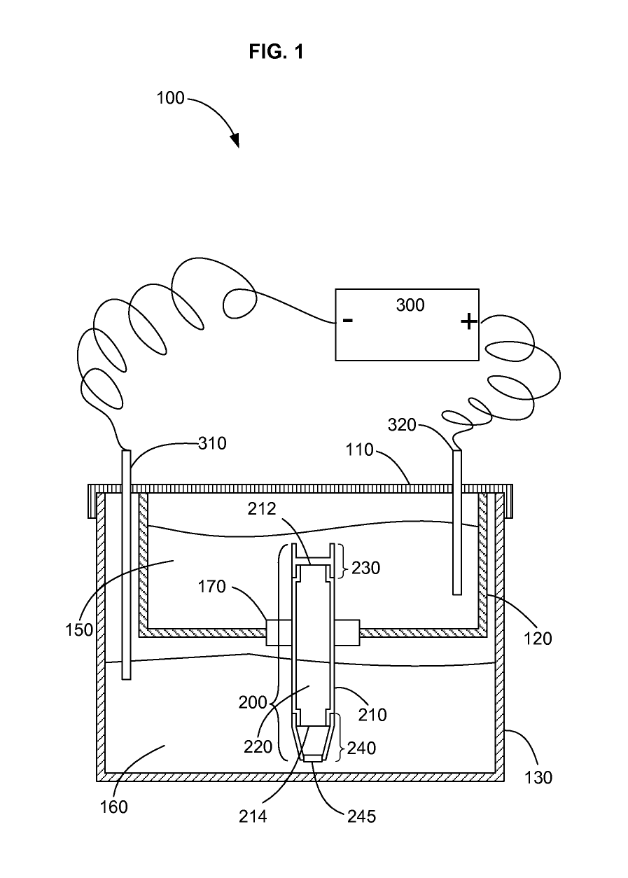

Disclosed is a device for the electrophoretic separation of free biomolecules from molecular complexes that include as constituents biomolecules of interest and the purification of such molecular complexes. Although a particular exemplary device is provided, the disclosed methods can be performed with other devices. With reference to FIG. 1 electrophoresis apparatus 100 includes upper chamber 120 that can contain solution 150, lower chamber 130 that can contain solution 160, and optionally lid 110. Solution 150 can be the same as, or different from, solution 160.

Upper chamber 120 and lower chamber 130 can of the same size, shape, and capacity, or of different sizes, shapes, and capacities as shown or even nested as depicted in FIG. 1. Upper chamber 120 can be mechanically and/or fluidly attached to lower chamber 130. Alternatively, upper chamber 120 can be mechanically and/or fluidly separate from lower chamber 130. In a one embodiment, upper chamber 120 and lower chamber 130 are mechanically and fluidly separate. In the embodiment shown in FIG. 1, electrophoresis device 100 has integrated tube gel 200, that includes column 210 which can optionally include removable cap 230 and removable collector 240. Column 210 can contain electrophoresis gel 220 that has proximal end 212 and distal end 214. Exemplary electrophoresis gels for use in the disclosed apparatus and methods are given below in section C. Tube gel 200 is fluidly connected to upper chamber 120 and lower chamber 130, permitting proximal end 212 of electrophoresis gel 220 to contact solution 150 and distal end 214 of electrophoresis gel 220 to contact solution 160.

In the embodiment depicted in FIG. 1, tube gel 200 is fluidly connected to upper chamber 120 and lower chamber 130 by passing through orifice 170. Orifice 170 is configured to retain tube gel 200 in position between upper chamber 120 and lower chamber 130, but allows tube gel 200 to be removed, for example to reverse the orientation of tube gel 200 such that proximal end 212 of gel 220 is now in contact with solution 160 and distal end 214 of gel 220 is now in contact with solution 150. Electrophoresis devices for use with the disclosed methods can be constructed from a variety of materials, such as glasses and plastics, for example polycarbonates, polystyrenes, polymethyl methacrylate, polyethylene polyfluoroethylene, polypropylene polyurethane, polyethylene terephthalate, polytetrafluoroethylene and the like.

Electrophoresis apparatus 100 also can include electricity source 300 (FIG. 1). The electricity source 300 includes first electrode 310 and second electrode 320. While a specific polarity of electricity source 300 is shown in FIG. 1, it is appreciated that the polarity can be reversed. Electrophoresis apparatus 100 can also include a detection device for detecting molecules separated by the apparatus electrophoresis apparatus 100, for example as they elute of distal end 212 of column 210 or alternatively proximal end 214 of electrophoresis gel 220. For example, electrophoresis apparatus 100 can be linked to a detection device such as an IR spectrophotometer, a UV spectrophotometer, a UV-VIS spectrophotometer, an HPLC machine, an NMR machine and the like.

Solutions 150 and 160 may be any solution that supports electrophoresis. In some embodiments, solution 150 is a salt solution. In some embodiments, solution 160 is a salt solution. In some embodiments, solution 150 is a buffer solution. In some embodiments, solution 160 is a buffer solution. Examples of salt solutions include: NaCl, KCl and the like. Examples of buffer solutions include: Tris, HEPES, MOPS, PBS, EDTA, Tris Buffered Saline, Tris-Borate-EDTA, TAE and the like.

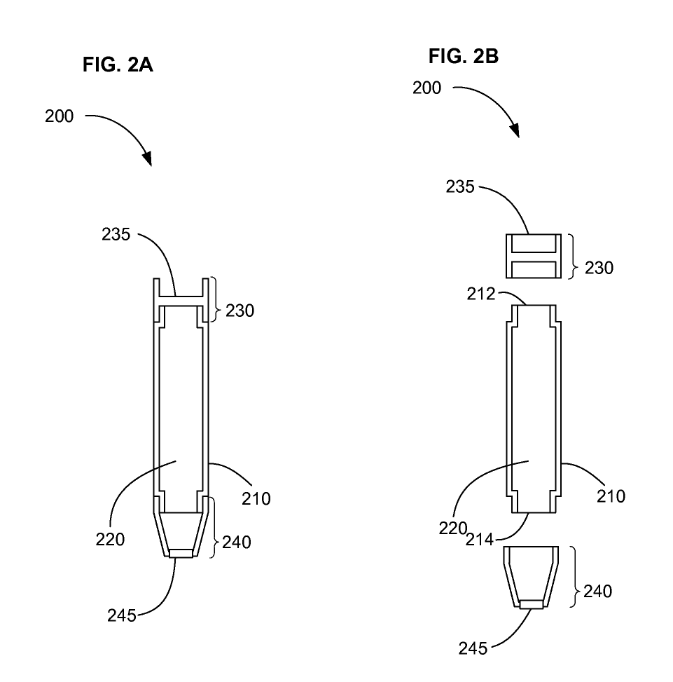

With reference to FIGS. 2A and 2B, column 200 can optionally include removable cap 230 and removable collector 240. Cap 230 includes well 235, which can be used to load samples into electrophoresis gel 220. Cap 230 is configured such that solution can pass through the cap, for example using a semi-permeable membrane. Collector 240 is configured such that solution can pass through the collector, for example using a semi-permeable membrane 245 with a molecular weight cut off that allows small molecules to pass through while retaining larger molecules, such as free biomolecules or biomolecules of interest. The molecular weight cut off can be from about 1 kilodalton (kDa) to about 300 kDa or greater, such as about 1 kDa to about 15 kDa, about 10 kDa to about 30 kDa, about 20 kDa to about 50 kDa, about 40 kDa to about 80 kDa, about 70 kDa to about 100 kDa, about 10 kDa to about 150 kDa, about 140 kDa to about 200 kDa, about 190 kDa to about 300 kDa, or even greater than about 300 kD.

Membranes with molecular weight cutoffs are available for AMICON® and MILLIPORE®, such as PLGC Ultracel RC 10 kDa; PLGC, Ultracel regenerated cellulose, 10 kDa; PBHK Biomax PES 300 kDa; PBMK, Biomax polyethersulfone, 300 kDa; PBHK Biomax PES100 kDa; PBHK, Biomax polyethersulfone, 100 kDa; PBTK Biomax PES 30 kDa, PBTK; PLHK Ultracel RC 100 kDa; PLHK, Ultracel regenerated cellulose, 100 kDa; PBHK Biomax PES 300 kDa; PBMK, Biomax polyethersulfone, 300 kDa; PLHK Ultracel RC 100 kDa; PLHK, Ultracel regenerated cellulose, 100 kDal; YM100 Ultracel RC 100 kDa 63.5 mm; YM-100, Ultracel regenerated cellulose, 100 kDa; PLAC Ultracel RC 1 kDa; PLAC, Ultracel regenerated cellulose, 1 kDa; PBHK Biomax PES 100 kDa; PBHK, Biomax polyethersulfone, 100 kDa; PBCC Biomax PES 5 kDa; PBCC, Biomax polyethersulfone, 5 kDa; PBHK Biomax PES 300 kDa; PBMK, Biomax polyethersulfone, 300 kDa; PBGC Biomax PES 10 kDa; PBGC, Biomax polyethersulfone, 10 kDa; YM10 Ultracel RC 10 kDa; YM-10, Ultracel regenerated cellulose, 10 kDa; YM1 Ultracel RC 1 kDa; M-1, Ultracel regenerated cellulose, 1 kDa; PLAC Ultracel RC 1 kDa; PLAC, Ultracel regenerated cellulose, 1 kDa; PLGC Ultracel RC 10 kDa; PLGC, Ultracel regenerated cellulose, 10 kDa; Ultracel RC 3 kDa; PLBC, Ultracel regenerated cellulose, 3 kDa; PM30 Amicon PES 30 kDa; M30, Amicon polyethersulfone, 30 kDa; PLGC Ultracel RC 10 kDa; PLGC; and Ultracel regenerated cellulose, 10 kDa. Other examples include Pierce SNAKESKIN® Pleated Dialysis Tubing, for example with molecular weight cutoffs of 3.5 kDa, 7 kDa MWCO, and 10 kDa.

### B. Methods of Electrophoretic Separation and Purification

Disclosed are methods for separating and purifying biomolecules of interest from a sample. The methods can include, loading a sample that includes the free biomolecules and the complexes that include the biomolecules of interest (which can include a detectable label) to the proximal end of the electrophoresis gel, for example in an electrophoresis device described in the preceding section. The sample is electrophoresed for a period of time sufficient for the free biomolecules to elute from the distal end of the electrophoresis gel, such that that the period of time is not long enough so that the complexes including the biomolecules of interest elute from the distal end of electrophoresis gel and are therefore retained in the electrophoresis gel. In this way, the complexes including the biomolecules of interest are separated from the free biomolecules. The time required for the free biomolecules to pass through the gel is dependent upon such factors as applied current, buffer conditions, gel concentration and gel length and the physical characteristics of the biomolecules. The free biomolecules can be discarded, for example by letting them elute from the distal end of the gel into surrounding buffer, or they can be collected, for example using a collector attached to the end of the gel, such as a collector with a semi-permeable membrane that allows buffer through, but retains the free biomolecules (exemplary semi-permeable membranes are described above).

To collect the complexes containing the biomolecules of interest, the current is reversed relative to the electrophoresis gel and the biomolecules remaining in the gel reverse their direction of travel relative to the electrophoresis gel. Reversing the current relative to the electrophoresis gel can be done by reversing the polarity of an electrophoresis apparatus, for example by reversing the direction of the current, while the gel remains static with respect to the electrophoresis apparatus, or reversing the orientation of the gel with respect to the applied electric current. The gel is then electrophoresed for a second period of time sufficient for the complexes of bound biomolecules of interest to elute from the proximal end of the electrophoresis gel where they can be collected, for example using a collector attached to the end of the gel, such as a collector with a semi-permeable membrane that allows buffer through, but retains the biomolecules of interest (exemplary semi-permeable membranes are described above). Reversing the direction of electrophoresis masks inhomogeneity in the complexes because the complexes that travel farther in one direction now must travel a longer distance in the reverse direction. This effectively cancels out the differences in the molecular weight, thereby masking any inhomogeneity between the complexes. By running the gel in one direction and then reversing the direction of electrophoresis, the biomolecules of interest in the molecular complex are both separated from the free biomolecules and concentrated.

Any biomolecule of interest can be purified using the disclosed methods. For example the methods disclosed herein can be used to purify nucleic acids of interest or polypeptides of interest. Thus, in some examples a biomolecule of interest is a polypeptide, such as a peptide or protein, for example a receptor, a receptor ligand, an antibody, an antigen, a soluble protein, or an insoluble protein and the like. In some examples polypeptides of interest include one or more antibodies. In some embodiments, polypeptides of interest include one or more antigens. In some examples, the biomolecules of interest are nucleic acid molecules, such as DNA, RNA or combinations thereof.

In some embodiments, the biomolecule of interest is a nucleic acid that is double stranded, single stranded, or a combination thereof. In some embodiments, the biomolecules of interest are partially double-stranded nucleic acid probes, such as those described in International Patent Publication WO 2008/147899, which is incorporated herein by reference in its entirety. International Patent Publication WO 2008/147899 claims the benefit of U.S. Provisional Application No. 60/939,826 which is also incorporated herein by reference in its entirety.

An application of the disclosed methods is the rapid and efficient determination of the sequence binding requirements for a given double-stranded nucleic acid binding protein, such as a double-stranded DNA binding protein, for example a transcription factor, such as an activated transcription factor. For example, by constructing a library of different double-stranded sequences and determining which sequences a particular transcription factor binds to it possible to rapidly identify the sequence requirements for a given transcription factor in a high throughput manner. Using the disclosed methods, such double-stranded sequences bound by transcription factors can be rapidly purified from unbound double-stranded sequences. Furthermore, because the methods masks the inhomogeneity present in the transcription factors, the method effectively eliminates the need for concentration, for example prior to applying the purified complexes to a micro array.

In some examples, a partially double-stranded nucleic acid probe includes two portions, a double-stranded portion and a single-stranded portion. The single-stranded portion includes a nucleotide sequence corresponding to an index sequence, such as but not limited to the index sequences described in International Patent Publication WO 2008/147899, which is incorporated herein by reference in its entirety. International Patent Publication WO 2008/147899 claims the benefit of U.S. Provisional Application No. 60/939,826, which is also incorporated herein by reference in its entirety.

The second portion of partially double-stranded nucleic acid probe is a double-stranded portion selected such that it contains one or more potential binding sites for double-stranded nucleic acid binding proteins, such as transcription factors, for example a partially double-stranded nucleic acid probe can contain 1, 2, 3, 4, 5, 6, 7, 8, 9, 10, or even more potential binding sites for double-stranded nucleic acid binding proteins, such as transcription factors, for example activated transcription factors. The double-stranded portion of the disclosed partially double-stranded nucleic acid probes are typically greater than about 8 nucleotide base pairs in length such as greater than about 8, about 9, about 10, about 11, about 12, about 13, about 14, about 15, about 20, about 25, about 30, about 35, about 40, about 45, about 50, about 60, about 70, about 80, about 90, about 100, about 120, about 140, about 160, about 180, about 200, about 250, about 300, or even greater than about 350 base pairs in length such as 8-50 nucleotides, 8-100 nucleotides, 8-200 nucleotides, 8-300 nucleotides, 8-500 nucleotides, or even greater than 500 nucleotides in length.

For the detection and/or isolation of biomolecules of interest, biomolecules of interest can include a label. The binding partner(s) of biomolecules of interest can include a label. Thus, in some embodiments, the biomolecules of interest and/or their binding partners are detectably labeled, with any isotopic or non-isotopic label known in the art. Non-isotopic labels can, for instance, include a fluorescent or luminescent molecule, biotin, an enzyme or enzyme substrate or a chemical. Isotopic labels can, for instance, include ³⁵S ¹⁴C, ³²P, ¹²⁵I, ³H isotopes and the like. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

The methods described above are depicted schematically in FIGS. 3A, 3B, 4, 5A and 5B, with reference to an exemplary tube gel (e.g. tube gel 200 of FIGS. 1-5). However, the method is not exclusive to tube gels, as gels of any configuration can be used to practice the disclosed methods. With reference to FIG. 3A, a sample is applied to the distal end 212 of electrophoresis gel 220. With reference to FIG. 3B as current is applied by the application of an electric field to electrophoresis gel 220, the biological macromolecules (such as the free biomolecules and complexes containing biomolecules of interest) are separated by the distance traveled through the gel as a function of time (the direction of migration is indicated). With reference to FIG. 3B (in which the free biomolecules have of a lower molecular weight) the free biomolecules will travel further as a function of time compared to molecular complexes (Complex 1 and Complex 2) that contain a biomolecule of interest because the molecular complexes have a larger molecular weights.

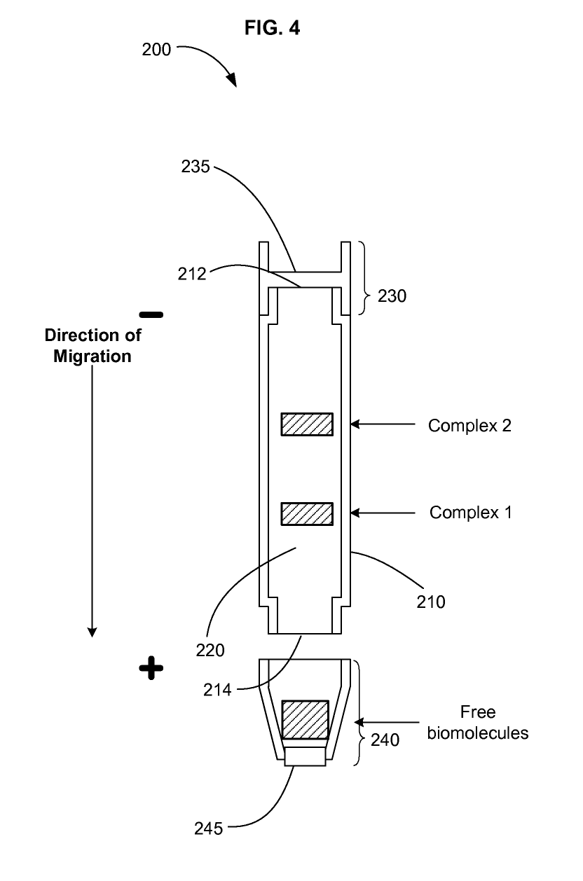

With reference to FIG. 4, the sample that included the free biomolecules and molecular complexes is electrophoresed for a period of time sufficient for the free biomolecules to elute off distal end 214 of electrophoresis gel 200, such that that the period of time is not long enough so that the complexes including the biomolecules of interest elute from distal end 212 of electrophoresis gel 220 and are retained in electrophoresis gel 200. The free biomolecules can be discarded, for example by letting them flow into the surrounding buffer (e.g. buffer 160 of FIG. 1), or they can be collected (as shown in FIG. 4), for example using collector 240, which can contain semi-permeable membrane 245 with a molecular weight cut off that allows the buffer to pass through but retains the free biomolecule (exemplary semi-permeable membranes are described above).

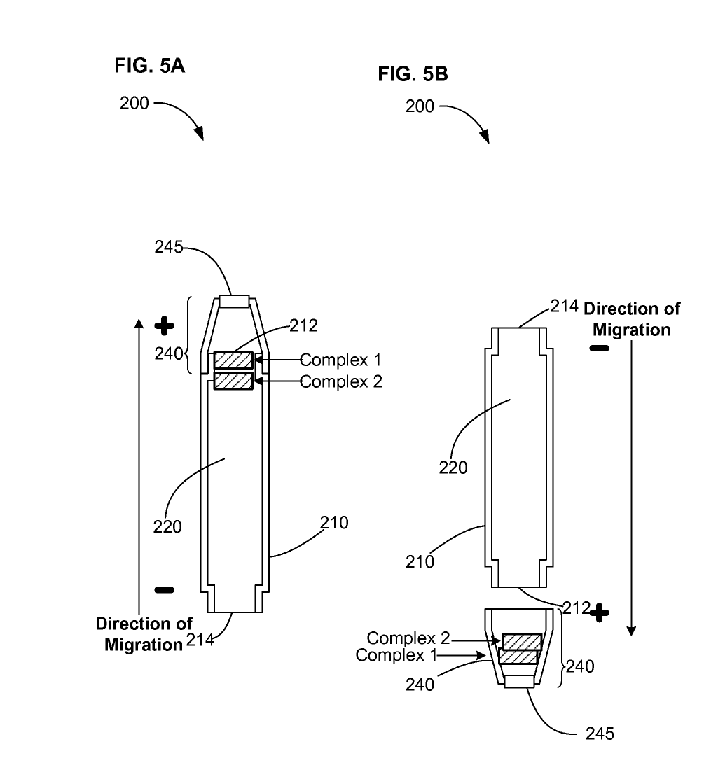

With reference to FIGS. 5A and 5B, the direction of electrophoresis is subsequently reversed, for example by reversing the orientation of column 200 as shown in FIG. 5A or by reversing the orientation as shown in FIG. 5B (note that in FIG. 5A the position of collector 240 is moved to proximal end 212 of tube gel 200). Complex 1 and Complex 2 travel in the opposite direction they originally traveled. Electrophoresis is continued for a period of time sufficient for the complexes of bound biomolecules of interest to elute from proximal end 212 of the electrophoresis gel 220. With reference to FIG. 5B the molecular complexes of bound biomolecules of interest (Complex 1 and Complex 2) can be collected, for example using collector 240, which can contain semi-permeable membrane 245 with a molecular weight cut off that allows the buffer to pass through but retains the molecular complexes (exemplary semi-permeable membranes are described above).

Biomolecules of interest and isolated molecular complexes can be collected and used for any purpose, for example for further analysis. In some examples, the biomolecules of interest are nucleic acid molecules, for example nucleic acid molecules to which transcription factors have bound. Thus, the methods can be used to separate nucleic acid molecules to which transcription factors have bound from nucleic acid molecules that have not been bound by transcription factors. The complexes containing such transcription factors can be collected and analyzed, for example using the methods described in International Patent Publication WO 2008/147899, which is incorporated herein by reference in its entirety. International Patent Publication WO 2008/147899 claims the benefit of U.S. Provisional Application No. 60/939,826 which is also incorporated herein by reference in its entirety. In other examples, the methods disclosed herein are used to separate protein complexes from free protein, for example a complex of antigen and antibody from free antigen or conversely from free antibody.

### C. Electrophoreses Gels

The methods and devices disclosed herein involve the use of an electrophoresis gel for the separation and/or purification of biomolecules of interest, such as biomolecules of interest in molecular complex. Electrophoresis gels suitable for the disclosed methods include gels composed of one or more polysaccharides (such as agarose), crossed linked polymers (a combination of cross-linkable monomers, for example acrylamide, that can be polymerized by a cross-linker, for example N,N′-methylenebisacrylamide) and combinations thereof.

The electrophoresis gels for use in the disclosed methods can be made from at least one polysaccharide, such as at least 1, at least 2, at least 3, at least 4, or more polysaccharides. In some embodiments, the polysaccharide is agarose or an agarose derivative, such as an agarose derivative which contains hydroxyethyl groups (for example, those disclosed in U.S. Pat. Nos. 3,956,273 and 4,319,975, or available from a commercial source, such as SEAKEM®, or NUSIEVE®), and the like. In other embodiments, the polysaccharide is cellulose or a cellulose derivative, such as hydroxyethyl cellulose, hydroxypropylmethyl, cellulose methyl cellulose, or the like. Agarose and cellulose are similar in that they are both linear polymers. They differ in their sugar constituents, in glycosidic linkages, and in the ability to form gels. While derivatized celluloses remain in solution at high and low temperatures, agarose polymers form thermally reversible gels. In other embodiments, the polysaccharide is galactomannan, dextran, starch, levan, glucan, mannan, xylan, or other polysaccharide. In specific embodiments, the polysaccharide is agarose, a derivative thereof, or a combination thereof.

Electrophoresis gels useful for the disclosed methods typically contain from about 0.1% polysaccharide to about 7% polysaccharide, such as about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0% about 5.1%, about 5.2%, about 5.3%, about 5.4%, about 5.5%, about 5.6%, about 5.7%, about 5.8%, about 5.9%, about 6.0% about 6.1%, about 6.2%, about 6.3%, about 6.5%, about 6.5%, about 6.6%, about 6.7%, about 6.8%, about 6.9%, or about 7.0% polysaccharide. The choice of percentage can be made based on factors such as the resolution required and the polysaccharide selected.

The electrophoresis gels made from cross-linked polymers for use in the disclosed methods can include the reaction mixture from the free radical polymerization reaction product of a mixture of at least one monomer, such as at least 1, at least 2, at least 3, at least 4, or more monomers, and at least one cross-linker, such as at least 1, at least 2, at least 3, at least 4 or more cross-linkers sufficient to cross-link the monomer. Suitable cross-linkable monomers include acrylamide, acrylamide derivatives (such as N-methylacrylamide, N,N-dimethylacrylamide, N-(hydroxymethyl)acrylamide, diacetonacrylamide, N-hydroxypropylacrylamide, and those disclosed in U.S. Pat. No. 5,185,466), other monomers, such as those disclosed in U.S. Pat. No. 5,840,877 (for example N-acryloyl-tris(hydroxymethyl)aminomethane, or N-acryloyl-1-amino-1-deoxy-D-galactitol), or a combination thereof. Other monomers that can be used in the disclosed methods include the acrylic monomers based on sugar alcohols disclosed in U.S. Pat. Nos. 5,185,466 and 5,202,007 and those disclosed in U.S. Pat. No. 5,319,046. It is contemplated that any of the monomers described herein can be used in the disclosed methods in any combination such that the resultant polymer gel is capable of separating biomolecules. In some embodiments, the monomer is acrylamide, N-methylacrylamide, N,N-dimethylacrylamide, N-(hydroxymethyl)acrylamide, diacetonacrylamide, N-hydroxypropylacrylamide, N-acryloyl-tris(hydroxymethyl)aminomethane, N-acryloyl-1-amino-1-deoxy-D-galactitol, or a combination thereof. In specific embodiments, the monomer is acrylamide. A cross-linking agent, such those described in Gelfi and Righetti Electrophoresis 2:213-219, 1981, or known to those of skill in the art can be used to form the crosslinked polymer gels for use in the disclosed methods. Crosslinked polymer gels suitable for the disclosed methods contain a cross-linker capable of cross-linking the monomers described herein. In some embodiments, the cross-linker is N,N′-methylenebisacrylamide, N,N′-propylenebisacrylamide, diacrylamide dimethylether, 1,2-diacrylamide ethyleneglycol, ethylenureabisacrylamide, ethylene diacrylate, N,N′-diallyltartardiamide, N,N′-bisacrylylcystamine, N,N′-1,2-dihydroxyethylene-bisacrylamide, N,N-bisacrylyl cystamine, trisacryloyl-hexahydrotriazine, dihydroxyethylene-bis-acrylamide, piperazine-di-acrylamide, or a combination thereof. In certain embodiments, the cross-linker is N,N′-methylenebisacrylamide (BIS). Cross linking agents are typically used in an amount of about 2% to about 30% by weight and preferably about 3% to about 15% by weight, based on the total weight of the monomer (such as acrylamide) and the cross-linking agent. Higher percentages of cross-linker typically result in gels that increase in opacity as the percentage of cross-linker increases.

In some examples, cross-linked polymer electrophoresis gels useful for the disclosed methods contain from about 0.1% cross-linked monomer to about 15% cross-linked monomer, such as about 0.1%, about 0.5%, about 1.0%, about 1.5%, about 2.0%, about 2.5%. about 3.0%, 3.5%, about 4.0%, about 4.5%, about 5.0%, about 6.0%, about 7.0%, about 7.5%, about 8.0%, about 8.5%, about 9.0%, about 9.5%, about 10.0%, 10.5%, about 11.0%, about 11.5%, about 12.0%. about 12.5%, about 13.0%, 13.5%, about 14.0%, about 14.5%, or about 15.0 cross-linked monomer. The choice of percentage can be made based on factors such as the resolution required and the monomer selected. For example, a percentage of monomer is chosen that is capable if resolving a biomolecule of interest from free biomolecules.

The cross-linking polymerization can be initiated in the presence of a peroxide and/or under irradiation of ultra-violet rays. The reaction can be further accelerated by heat and irradiation with ultra-violet rays. As the polymerization catalyst, a known low temperature-polymerization initiator can be used, such as those described in Gelfi and Righetti Electrophoresis 2:213-219, 1981, Gelfi and Righetti Electrophoresis 2:220-228, 1981; and Modern Electrophoresis edited by Aoki and Nagai (Hirokawa Shoten, 1973). Examples of initiators include a mixture of beta-dimethylaminopropionitrile and ammonium peroxodisulfate, a mixture of N,N,N′,N′-tetramethylethylenediamine (TEMED) and ammonium peroxodisulfate, a mixture of TEMED and riboflavin, a combination of a mixture of TEMED, riboflavin and hydrogen peroxide, and irradiation with ultra-violet rays. The most common system of catalytic initiation involves the production of free oxygen radicals by ammonium persulfate (APS) in the presence of the tertiary aliphatic amine TEMED.

The gel-based electrophoretic embodiments of this disclosure can be carried out in any suitable format, for example in standard-sized gels, tubes, minigels, strips, capillaries, and gels designed for use with microtiter plates and other high throughput (HTS) applications, and the like. Formats for gels include those described in U.S. Pat. Nos. 5,578,180; 5,922,185; 6,057,106; 6,059,948; 6,096,182; 6,143,154; 6,162,338; 6,562,213, U.S. Patent Publications 20020134680, 20030127330 and 20030121784; and published PCT Application Nos. WO 95/27197, WO 99/37813, WO 02/18901 and WO 02/071024.

### D. Samples

Appropriate samples for use in the methods disclosed herein include any conventional biological sample for which separation and/or purification of biomolecules of interest is desired. In some examples, samples can be samples of purified biomolecules, for example biomolecules recombinantly or synthetically produced or purified from a biological source such as an organism. In some examples, samples include those obtained from, excreted by or secreted by any living organism (whether the organism is live or dead), such as a prokaryotic organism or a eukaryotic organism including without limitation, multicellular organisms (such as plants and animals, including samples from a healthy or apparently healthy human subject or a human patient affected by a condition or disease to be diagnosed or investigated, such as cancer), clinical samples obtained from a human or veterinary subject, for instance blood or blood-fractions, biopsied tissue. Standard techniques for acquisition of such samples are available. See, for example Schluger et al., J. Exp. Med. 176:1327-33 (1992); Bigby et al., Am. Rev. Respir. Dis. 133:515-18 (1986); Kovacs et al., NEJM 318:589-93 (1988); and Ognibene et al., Am. Rev. Respir. Dis. 129:929-32 (1984). Biological samples can be obtained from any organ or tissue (including a biopsy or autopsy specimen, such as a tumor biopsy) or can comprise a cell (whether a primary cell or cultured cell) or medium conditioned by any cell, tissue or organ. In some embodiments, a biological sample is a nuclear extract. Nuclear extract contains many of the proteins contained in the nucleus of a cell, and includes for example transcription factors, such as activated transcription factors. Methods for obtaining a nuclear extract are well known in the art and can be found for example in Dignam, Nucleic Acids Res, 11; 11(5):1475-89 1983.

The following examples are provided to illustrate particular features of certain embodiments. However, the particular features described below should not be construed as limitations on the scope of the disclosure, but rather as examples from which equivalents will be recognized by those of ordinary skill in the art.

### EXAMPLES

### Example 1

### Gel Shift Analysis of Sp-1 Protein Binding to Partially Double-Stranded Nucleic Acid Probes

IRDye 700 labeled oligos (YZ-7f, YZ-9f, YZ-11f, YZ-7b, YZ-9b and YZ-11b, see Table 1) were synthesized at Li-cor, Inc and annealed to be IRDye 700 labeled double strand DNA probes (YZ-7, YZ-9 and YZ-11). The double-stranded nucleic acid probes were mixed with SP-1 protein (Promega) under conditions that permit the protein to bind to the double-stranded nucleic acid and subjected to polyacrylamide gel electrophoresis.

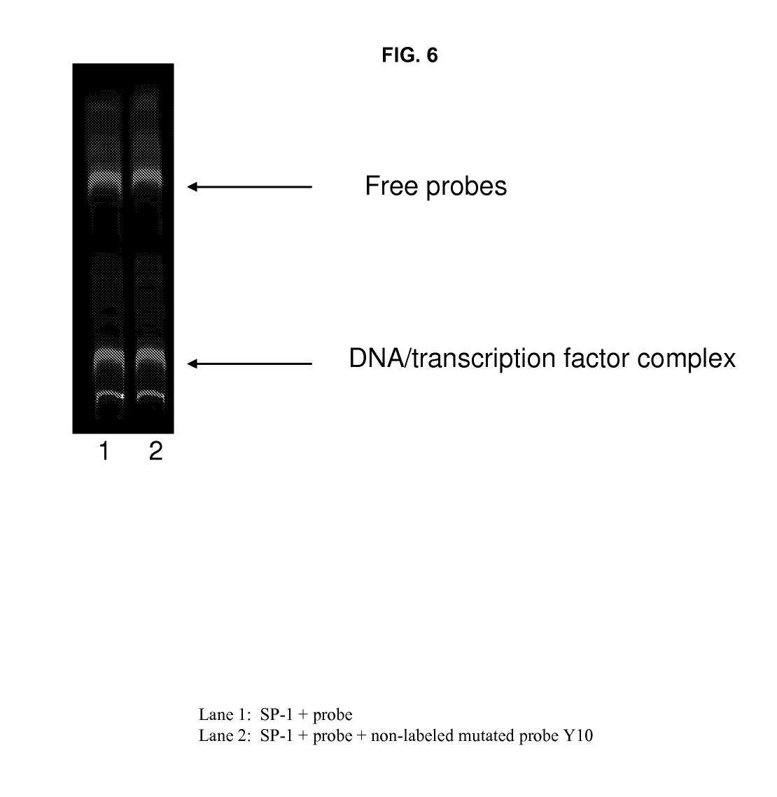

The gels were imaged, the results of which are shown in FIG. 6. With reference to FIG. 6 recombinant SP-1 is able to bind to the YZ9 partially double-stranded nucleic acid probe that included the SP-1 binding sequence. This result demonstrates that the SP-1 transcription factor bound double-stranded nucleic acid probes can be separated by gel electrophoresis.

TABLE 1

| | | |
|---|---|---|
| | | SEQ ID |
| Name | Sequence | NO: |

Probes for NF-kb

| | | |
|---|---|---|
| YZ-7f | TCC TAG CTT CAG AGG GGA CTT TCC GAG | 1 |
| | AGG ACC TGA AGA AAT GGT TTT GAA TCA T | |
| YZ-7b | CCT CTC GGA AAG TCC CCT CTG AAG CTA | 2 |
| | GGA | |

Probes for Sp1

| | | |
|---|---|---|
| YZ-9f | AGC TTA TTC GAT CGG GGC GGG GCG AGC | |
| | GAA GTT ATC CCA ACT TCG AAT CTC ATT T | 3 |
| YZ-9b | TTC GCT CGC CCC GCC CCG ATC GAA TAA | 4 |
| | GCT | |

Probes for ER-alpha

| | | |
|---|---|---|
| YZ-11f | CCT GCC AGG TCG CGC TGC CCT CCT TCT | 5 |
| | ACC ATG CCT TAG GAG AAT TGT TTT GTT T | |
| YZ-11b | GGT AGA AGG AGG GCA GCG CGA CCT GGC | 6 |
| | AGG | |

### Example 2

### Microarray Analysis of Partially Double-Stranded Nucleic Acid Probes Selected as Sp-1 Binding Sites

For microarray analysis, 5′-end cyanine (Cy3) labeled oligonucleotides (YZ-7f, YZ-9f and YZ-11f) and unlabeled oligonucleotides (YZ-7b, YZ-9b and YZ-11b) were synthesized at Integrated DNA Technologies, Inc. and annealed to yield Cy3-labeled double strand DNA probes. The probes include a double-stranded transcription factor binding motif and a unique single strand tag that can hybridize to a specific oligonucleotide printed on a microarray slide.

The Sp1 protein was mixed with a group of Cy3 labeled probes (YZ-7, YZ-9 and YZ-11) at room temperature for 30 minutes and then the protein/DNA complex was separated on the polyacrylamide column using the separation method described in Example 1. The collected protein/DNA complex was concentrated, the buffer changed to 5×SSC, 0.1% SDS, and the DNA hybridized to a microarray slide containing oligonucleotide DNA sequences shown in Table 2. Small amounts of YZ-2 and YZ-4 were added (shown in Table 3 and complementary to the sequences of YZ-1 and YZ-3). These sequences, shown in Table 3, serve as a positive control and reference signal. Only the Sp1 and control probes yielded positive signals (see Table 4). This demonstrates that Sp1/DNA complexes can be separated and collected by the disclosed method and apparatus described, and then identified using microarray technology. The microarray results (shown in Table 4) was consistent with the result from the gel shift assay.

TABLE 2
Oligonucleotide sequences printed
on slide for microarray analysis

| | | SEQ ID |
|---|---|---|
| Name | Sequence | NO: |
| YZ1 | TGG TTG TCA TCT GGG ATT ACT TTT A | 7 |
| YZ3 | GGG TTT TTT TTT TCC CGT TTT TTT TGG G | 8 |
| Y-5t | ACA TAG CAT CCC TCA AAC TAT ACA A | 9 |
| Y-7t | AGC TTT GAA TGG TCT AGT CAA AAA A | 10 |
| Y-8t | TCT ACA TTC AGG ATA AGA TTT GGC T | 11 |
| Y-9t | AAA TGA GAT TCG AAG TTG GGA TAA C | 12 |
| Y-11t | AAA CAA AAC AAT TCT CCT AAG GCA T | 13 |
| Y-12t | CAA AGA AAA GGG GCT ACA CAA TTA T | 14 |
| Y-14 | ATG ATT CAA AAC CAT TTC TTC AGG T | 15 |
| Y-15 | TTA AAC ATT GTG TGT TAA CAC CTG T | 16 |
| y-16 | GGT TCA TAG ATG GTC AGT TTT GTA C | 17 |
| y-17 | AGT GTT CCC AAT CTG AAA TTC AAA A | 18 |
| y-18 | GTC CTG TTA TTC TGA CTA CAG TTC T | 19 |
| y-19 | CTG GAG TTA CAG TTT TCA ATC TGT C | 20 |
| y-20 | AAG CTA CGG TAC CAG TAA TTA GAT G | 21 |
| y-21 | TTG GAC ACT ATC TTG ATC AGA AGA G | 22 |
| y-22 | TCC ATG CAC ATT TAC AAT ATT GAG G | 23 |
| y-23 | GTT TTA GTT CCG TTC TCG TTT TCT T | 24 |
| y-24 | GCT AGA AAA ATA GGG CTG GAT CTT A | 25 |
| y-25 | CAT ATT GAT TGG TGA AAT TGG TGG T | 26 |
| y-26 | AAG TTG TTT GAG GCA AAT TAA CTG T | 27 |
| y-27 | TCT ATT GAA TTC GGA ACT GTC CTT T | 28 |
| y-28 | AAA GCC TCT TTT CGA ATA AAG CAA A | 29 |
| y-29 | AAT TGT TTT GTT TCA CAA AAG CTG G | 30 |

TABLE 3
Oligonucleotide sequences functioning as
positive control and reference signal

| | | SEQ ID |
|---|---|---|
| name | Sequence | NO: |
| YZ2 | TAA AAG TAA TCC CAG ATG ACA ACC A | 31 |
| YZ4 | CCC AAA AAA AAC GGG AAA AAA AAA ACC | 32 |

TABLE 4
Identification of Sp1/DNA complex by microarray

| Probe | Signal |
|---|---|
| Y-5t | 0 |
| Y-7t | 0 |
| Y-8t | 0 |
| Y-9t | 936.1735 |
| YZ2 | 2051.364 |
| YZ4 | 498.5943 |
| Y-11t | 0 |
| Y-12t | 0 |
| Y-14 | 0 |
| Y-15 | 0 |
| Y-16 | 0 |
| Y-17 | 0 |
| Y-18 | 0 |
| Y-19 | 0 |
| Y-20 | 0 |
| Y-21 | 0 |
| Y-22 | 0 |
| Y-23 | 0 |
| Y-24 | 0.4779 |
| Y-25 | 0 |
| Y-26 | 0 |
| Y-27 | 0 |
| Y-28 | 0 |
| Y-29 | 0 |

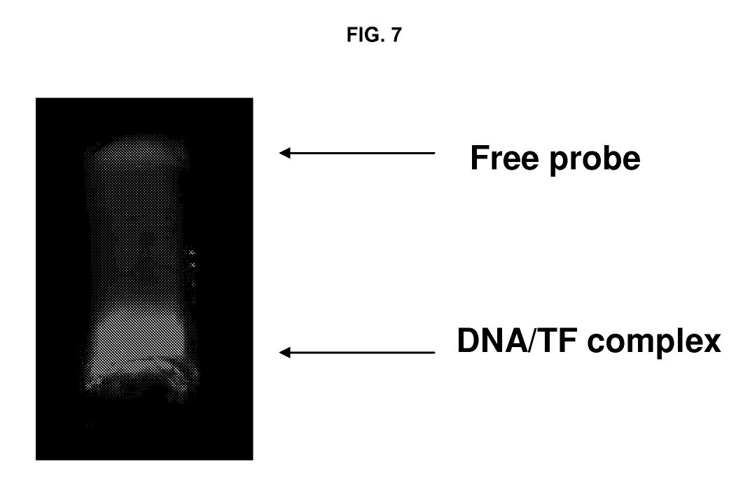

A similar result is obtained using recombinant estrogen receptor alpha (ER-alpha) protein. ER-alpha is obtained from INVITROGEN® and mixed with YZ11 (its specific probe) labeled with IR Dye 700. The mixture was then loaded on the column gel and run for 30 minutes (FIG. 7).

### Example 3

### Microarray Analysis of Partially Double-Stranded Nucleic Acid Probes Selected as Sp-1 Binding Site and Concentrated with Reversed Electrophoresis

5′-end cyanine (Cy3) labeled oligonucleotides (YZ-7f, YZ-9f and YZ-11f) and unlabeled oligonucleotides (YZ-7b, YZ-9b and YZ-11b) are synthesized at Integrated DNA Technologies, Inc. and are annealed to yield Cy3-labeled double strand DNA probes. The probes include a double-stranded transcription factor binding motif and a unique single strand tag that can hybridize to a specific oligonucleotide printed on a microarray slide.

The Sp1 protein is mixed with a group of Cy3 labeled probes (YZ-7, YZ-9 and YZ-11) at room temperature for 30 minutes and then the protein/DNA complex is separated from unbound probes on the polyacrylamide column using for a period of time sufficient for the unbound probes to elute from the distal end of the electrophoresis gel. The orientation of the column is reversed and the sample is electrophoreses for a period of time sufficient for the protein/DNA to elute from the proximal end of the electrophoresis gel. The protein/DNA complexes are collected. The buffer is changed to 5×SSC, 0.1% SDS, and the DNA is hybridized to a microarray slide containing oligonucleotide DNA sequences shown in Table 2. Small amounts of YZ-2 and YZ-4 are added (shown in Table 3 and complementary to the sequences of YZ-1 and YZ-3) as a positive control and reference signal.

While this disclosure has been described with an emphasis upon particular embodiments, it will be obvious to those of ordinary skill in the art that variations of the particular embodiments can be used, and it is intended that the disclosure may be practiced otherwise than as specifically described herein. Features, characteristics, compounds, chemical moieties, or examples described in conjunction with a particular aspect, embodiment, or example of the disclosure are to be understood to be applicable to any other aspect, embodiment, or example of the disclosure. Accordingly, this disclosure includes all modifications encompassed within the spirit and scope of the disclosure as defined by the following claims.

1. A method for electrophoretic separation of free biomolecules from molecular complexes comprising biomolecules of interest, wherein inhomogeneity in the molecular complexes is masked, the method comprising:
electrophoresing a sample comprising free biomolecules and the molecular complexes comprising the biomolecules of interest in an electrophoresis gel for a period of time sufficient for the free biomolecules to elute off a distal end of the electrophoresis gel, wherein the molecular complexes comprising the biomolecules of interest are retained in the electrophoresis gel, thereby separating the molecular complexes comprising the biomolecules of interest from the free biomolecules; and
reversing direction of electrophoresis and electrophoresing the sample for a period of time sufficient for the molecular complexes of bound biomolecules of interest to elute from a proximal end of the electrophoresis gel, wherein reversing the direction of electrophoresis masks inhomogeneity in the molecular complexes comprising biomolecules of interest.

2. The method of claim 1, further comprising loading a sample comprising the free biomolecules and the molecular complexes comprising the biomolecules of interest to a proximal end of the electrophoresis gel.

3. The method of claim 1, further comprising collecting the molecular complexes comprising biomolecules of interest.

4. The method of claim 1, further comprising collecting the free biomolecules.

5. The method of claim 1, wherein reversing direction of electrophoresis comprises reversing the orientation of the electrophoresis gel in an electrophoresis apparatus.

6. The method of claim 1, wherein reversing direction of electrophoresis comprises reversing the polarity of an electrophoresis apparatus.

7. The method of claim 1, wherein the electrophoresis gel comprises a polysaccharide.

8. The method of claim 7, wherein the polysaccharide comprises hydroxyethyl cellulose, hydroxypropylmethyl cellulose, methyl cellulose, agarose, hydroxyethyl agarose, galactomannan, dextran or a combination thereof.

9. The method of claim 8, wherein the polysaccharide comprises agarose.

10. The method of claim 1, wherein the electrophoresis gel comprises a crosslinked polymer.

11. The method of claim 10, wherein the cross-linked polymer comprises a free radical polymerization reaction product of a mixture comprising at least one monomer and at least one cross-linker sufficient to cross-link the monomer, wherein the monomer comprises acrylamide, N-methylacrylamide, N,N-dimethylacrylamide, N-(hydroxymethyl)acrylamide, diacetonacrylamide, N-hydroxypropylacrylamide N-hydroxypropylacrylamide, N-acryloyl-tris(hydroxymethyl)aminomethane, N-acryloyl-1-amino-1-deoxy-D-galactitol, or a combination thereof, and the cross-linker comprises N,N′-methylenebisacrylamide, N,N′-propylenebisacrylamide, diacrylamide dimethylether, 1,2-diacrylamide ethyleneglycol, ethylenureabisacrylamide, ethylene diacrylate, N,N′-diallyltartardiamide, N,N′-bisacrylylcystamine, N,N′-1,2-dihydroxyethylene-bisacrylamide, N,N-bisacrylyl cystamine, trisacryloyl-hexahydrotriazine, dihydroxyethylene-bis-acrylamide, piperazine-di-acrylamide, or a combination thereof.

12. The method of claim 11 wherein the cross-linked polymer comprises cross-linked acrylamide.

13. The method of claim 1, wherein the biomolecules of interest in the sample comprise a detectable label.

14. The method of claim 1, wherein the biomolecules of interest in the sample comprise nucleic acids of interest or polypeptides of interest.

15. The method of claim 14, wherein the polypeptides of interest comprise antibodies.

16. The method of claim 14, wherein the polypeptides of interest comprise antigens.

17. The method of claim 14, wherein the nucleic acids of interest are double stranded, single stranded, or a combination thereof.

18.-22. (canceled)

23. A method for masking inhomogeneity of one or both members of a specific binding pair comprising a biomolecule of interest during an electrophoretic purification of the specific binding pair comprising a biomolecule of interest, the method comprising:
electrophoresing in an electrophoresis gel a sample comprising the specific binding pair comprising a biomolecule of interest and the unbound biomolecules for a period of time sufficient for the unbound biomolecules to elute from a distal end of the electrophoresis gel, and wherein the specific binding pair comprising the biomolecule of interest is retained in the electrophoresis gel, thereby separating the unbound biomolecules from the specific binding pair comprising the biomolecule of interest; and
reversing direction of electrophoresis and electrophoresing the sample for a period of time sufficient for the specific binding pair comprising a biomolecule of interest to elute from a proximal end of the electrophoresis gel, wherein reversing the direction of electrophoresis masks inhomogeneity in the specific binding pair comprising a biomolecule of interest.

24. The method of claim 23, further comprising loading a sample comprising the specific binding pair comprising a biomolecule of interest and unbound biomolecules to a proximal end of the electrophoresis gel.

25. The method of claim 23, further comprising collecting the specific binding pair comprising the biomolecule of interest.

26. The method of claim 23, further comprising collecting the free biomolecules.

27. The method of claim 23, wherein reversing direction of electrophoresis comprises reversing the orientation of the electrophoresis gel in an electrophoresis apparatus.

28. The method of claim 23, wherein reversing direction of electrophoresis comprises reversing the polarity of an electrophoresis apparatus.

29. The method of claim 23, wherein the electrophoresis gel comprises a polysaccharide.

30. The method of claim 29, wherein the polysaccharide comprises hydroxyethyl cellulose, hydroxypropylmethyl cellulose, methyl cellulose, agarose, hydroxyethyl agarose, galactomannan, dextran or a combination thereof.

31. The method of claim 30, wherein the polysaccharide comprises agarose.

32. The method of claim 23, wherein the electrophoresis gel comprises a crosslinked polymer.

33. The method of claim 32, wherein the cross-linked polymer comprises the free radical polymerization reaction product of a mixture comprising at least one monomer and at least one cross-linker sufficient to cross-link the monomer, wherein the monomer comprises acrylamide, N-methylacrylamide, N,N-dimethylacrylamide, N-(hydroxymethyl)acrylamide, diacetonacrylamide, N-hydroxypropylacrylamide N-hydroxypropylacrylamide, N-acryloyl-tris(hydroxymethyl)aminomethane, N-acryloyl-1-amino-1-deoxy-D-galactitol, or a combination thereof, and the cross-linker comprises N,N′-methylenebisacrylamide, N,N′-propylenebisacrylamide, diacrylamide dimethylether, 1,2-diacrylamide ethyleneglycol, ethylenureabisacrylamide, ethylene diacrylate, N,N′-diallyltartardiamide, N,N′-bisacrylylcystamine, N,N′-1,2-dihydroxyethylene-bisacrylamide, N,N-bisacrylyl cystamine, trisacryloyl-hexahydrotriazine, dihydroxyethylene-bis-acrylamide, piperazine-di-acrylamide, or a combination thereof.

34. The method of claim 32, wherein the cross-linked polymer comprises cross-linked acrylamide.

35. The method of claim 23, wherein the biomolecules of interest in the sample comprise a detectable label.

36. The method of claim 23, wherein the biomolecules of interest in the sample comprise nucleic acids of interest or polypeptides of interest.

37. The method of claim 36, wherein the polypeptides of interest comprise antibodies.

38. The method of claim 36, wherein the polypeptides of interest comprise antigens.

39. The method of claim 36, wherein the nucleic acids of interest are double stranded, single stranded, or a combination thereof.

40.-44. (canceled)


SEQUENCE LISTING (32 sequences)

SEQ ID NO: 1 - DNA, 55 bp, Artificial sequence
  Synthetic oligonucleotide probe.
tcctagcttcagaggggactttccgagaggacctgaagaaatggttttgaatcat

SEQ ID NO: 2 - DNA, 30 bp, Artificial sequence
  Synthetic oligonucleotide probe.
cctctcggaaagtcccctctgaagctagga

SEQ ID NO: 3 - DNA, 55 bp, Artificial sequence
  Synthetic oligonucleotide probe.
agcttattcgatcggggcggggcgagcgaagttatcccaacttcgaatctcattt

SEQ ID NO: 4 - DNA, 30 bp, Artificial sequence
  Synthetic oligonucleotide probe.
ttcgctcgccccgccccgatcgaataagct

SEQ ID NO: 5 - DNA, 55 bp, Artificial sequence
  Synthetic oligonucleotide probe.
cctgccaggtcgcgctgccctccttctaccatgccttaggagaattgttttgttt

SEQ ID NO: 6 - DNA, 30 bp, Artificial sequence
  Synthetic oligonucleotide probe.
ggtagaaggagggcagcgcgacctggcagg

SEQ ID NO: 7 - DNA, 25 bp, Artificial sequence
  Synthetic oligonucleotide probe.
tggttgtcatctgggattactttta

SEQ ID NO: 8 - DNA, 28 bp, Artificial sequence
  Synthetic oligonucleotide probe.
gggttttttttttcccgttttttttggg

SEQ ID NO: 9 - DNA, 25 bp, Artificial sequence
  Synthetic oligonucleotide probe.
acatagcatccctcaaactatacaa

SEQ ID NO: 10 - DNA, 25 bp, Artificial sequence
  Synthetic oligonucleotide probe.
agctttgaatggtctagtcaaaaaa

SEQ ID NO: 11 - DNA, 25 bp, Artificial sequence
  Synthetic oligonucleotide probe.
tctacattcaggataagatttggct

SEQ ID NO: 12 - DNA, 25 bp, Artificial sequence
  Synthetic oligonucleotide probe.
aaatgagattcgaagttgggataac

SEQ ID NO: 13 - DNA, 25 bp, Artificial sequence
  Synthetic oligonucleotide probe.
aaacaaaacaattctcctaaggcat

SEQ ID NO: 14 - DNA, 25 bp, Artificial sequence
  Synthetic oligonucleotide probe.
caaagaaaaggggctacacaattat

SEQ ID NO: 15 - DNA, 25 bp, Artificial sequence
  Synthetic oligonucleotide probe.
atgattcaaaaccatttcttcaggt

SEQ ID NO: 16 - DNA, 25 bp, Artificial sequence
  Synthetic oligonucleotide probe.
ttaaacattgtgtgttaacacctgt

SEQ ID NO: 17 - DNA, 25 bp, Artificial sequence
  Synthetic oligonucleotide probe.
ggttcatagatggtcagttttgtac

SEQ ID NO: 18 - DNA, 25 bp, Artificial sequence
  Synthetic oligonucleotide probe.
agtgttcccaatctgaaattcaaaa

SEQ ID NO: 19 - DNA, 25 bp, Artificial sequence
  Synthetic oligonucleotide probe.
gtcctgttattctgactacagttct

SEQ ID NO: 20 - DNA, 25 bp, Artificial sequence
  Synthetic oligonucleotide probe.
ctggagttacagttttcaatctgtc

SEQ ID NO: 21 - DNA, 25 bp, Artificial sequence
  Synthetic oligonucleotide probe.
aagctacggtaccagtaattagatg

SEQ ID NO: 22 - DNA, 25 bp, Artificial sequence
  Synthetic oligonucleotide probe.
ttggacactatcttgatcagaagag

SEQ ID NO: 23 - DNA, 25 bp, Artificial sequence
  Synthetic oligonucleotide probe.
tccatgcacatttacaatattgagg

SEQ ID NO: 24 - DNA, 25 bp, Artificial sequence
  Synthetic oligonucleotide probe.
gttttagttccgttctcgttttctt

SEQ ID NO: 25 - DNA, 25 bp, Artificial sequence
  Synthetic oligonucleotide probe.
gctagaaaaatagggctggatctta

SEQ ID NO: 26 - DNA, 25 bp, Artificial sequence
  Synthetic oligonucleotide probe.
catattgattggtgaaattggtggt

SEQ ID NO: 27 - DNA, 25 bp, Artificial sequence
  Synthetic oligonucleotide probe.
aagttgtttgaggcaaattaactgt

SEQ ID NO: 28 - DNA, 25 bp, Artificial sequence
  Synthetic oligonucleotide probe.
tctattgaattcggaactgtccttt

SEQ ID NO: 29 - DNA, 25 bp, Artificial sequence
  Synthetic oligonucleotide probe.
aaagcctcttttcgaataaagcaaa

SEQ ID NO: 30 - DNA, 25 bp, Artificial sequence
  Synthetic oligonucleotide probe.
aattgttttgtttcacaaaagctgg

SEQ ID NO: 31 - DNA, 25 bp, Artificial sequence
  Synthetic oligonucleotide probe.
taaaagtaatcccagatgacaacca

SEQ ID NO: 32 - DNA, 27 bp, Artificial sequence
  Synthetic oligonucleotide probe.
cccaaaaaaaacgggaaaaaaaaaacc